(19) United States
(12) Patent Application Publication
(10) Pub. No.: US 2018/0049970 A1
(43) Pub. Date: Feb. 22, 2018

(54) HAIR CARE COMPOSITIONS COMPRISING METATHESIZED UNSATURATED POLYOL ESTERS

(71) Applicant: The Procter & Gamble Company, Cincinnati, OH (US)

(72) Inventors: Qing STELLA, Cincinnati, OH (US); Beth Ann SCHUBERT, Maineville, OH (US); Michael Stephen MAILE, Maineville, OH (US)

(21) Appl. No.: 15/655,075

(22) Filed: Jul. 20, 2017

Related U.S. Application Data
(60) Provisional application No. 62/376,617, filed on Aug. 18, 2016.

(51) Int. Cl. A61K 8/92; A61K 8/02; A61K 8/41; A61K 8/34; A61K 8/88; A61Q 5/12

(52) U.S. Cl. CPC A61K 8/922; A61K 8/0216; A61K 8/416; A61K 2800/5922; A61K 8/345; A61K 8/88; A61Q 5/12; A61K 8/34

(57) ABSTRACT

Disclosed are hair care compositions, such as conditioners, containing a metathesized unsaturated polyol ester; and a gel matrix phase comprising one or more high melting point fatty compounds, a cationic surfactant system an aqueous carrier. The oligomers provide beneficial hair benefits. Also disclosed are methods of using the hair care compositions.

### FIELD OF THE INVENTION

The present invention relates to a hair care composition containing a gel matrix and an oligomer derived from metathesis of unsaturated polyol esters, and methods of using the same.

### BACKGROUND OF THE INVENTION

Human hair becomes soiled due to its contact with the surrounding environment and from the sebum secreted by the scalp. The soiling of hair causes it to have a dirty feel and an unattractive appearance.

Shampooing cleans the hair by removing excess soil and sebum. However, shampooing can leave the hair in a wet, tangled, and generally unmanageable state. Once the hair dries, it is often left in a dry, rough, lusterless, or frizzy condition due to removal of the hair's natural oils.

A variety of approaches have been developed to alleviate these after-shampoo problems. One approach is the application of hair shampoos which attempt to both cleanse and condition the hair from a single product.

In order to provide hair conditioning benefits in a cleansing shampoo base, a wide variety of conditioning actives have been proposed. However, including active levels of conditioning agents in shampoos may result in rheology and stability issues, creating consumer trade-offs in cleaning, lather profiles, and weigh-down effects. Additionally, the rising costs of silicone and the petroleum based nature of silicone have minimized silicone's desirability as a conditioning active.

Based on the foregoing, there is a need for a conditioning active which can provide conditioning benefits to hair and can replace, or be used in combination with silicone, or other conditioning actives, to maximize the conditioning activity of hair care compositions. Additionally, there is a desire to find a conditioning active which can be derived from a natural source, thereby providing a conditioning active derived from a renewable resource. There is also a desire to find a conditioning active that is both derived from a natural source and leads to a stable product comprising a micellar surfactant system.

### SUMMARY OF THE INVENTION

In one aspect, the present invention is directed to hair care composition comprising: (a) from about 0.05% to about 15%, by weight of said hair care composition, of one or more metathesized unsaturated polyol esters, said metathesized unsaturated polyol ester having one or more of the following properties: (i) a free hydrocarbon content, based on total weight of metathesized unsaturated polyol ester, of from about 0% to about 5%; (ii) a weight average molecular weight of from about 5,000 Daltons to about 50,000 Daltons; (iii) an iodine value of from about 30 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

In another aspect, the present invention is directed to hair care composition comprising: a) a metathesized unsaturated polyol ester, said metathesized unsaturated polyol ester having a weight average molecular weight of from about 2,000 Daltons to about 50,000 Daltons; and one or more of the following properties: (i) a free hydrocarbon content, based on total weight of metathesized unsaturated polyol ester, of from about 0% to about 5%; or (ii) an iodine value of from about 8 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

The present invention also is directed to a method for cleansing hair with an effective amount of the hair care composition described above.

These and other features, aspects, and advantages of the present invention will become evident to those skilled in the art from a reading of the present disclosure.

### DETAILED DESCRIPTION OF THE INVENTION

### Definitions

The terms “natural oils,” “natural feedstocks,” or “natural oil feedstocks” may refer to oils derived from plants or animal sources. The term “natural oil” includes natural oil derivatives, unless otherwise indicated. The terms also include modified plant or animal sources (e.g., genetically modified plant or animal sources), unless indicated otherwise. Examples of natural oils include, but are not limited to, vegetable oils, algae oils, fish oils, animal fats, tall oils, derivatives of these oils, combinations of any of these oils, and the like. Representative non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, pennycress oil, camelina oil, and castor oil. Representative non-limiting examples of animal fats include lard, tallow, poultry fat, yellow grease, and fish oil. Tall oils are by-products of wood pulp manufacture.

The term “natural oil derivatives” refers to derivatives thereof derived from natural oil. The methods used to form these natural oil derivatives may include one or more of addition, neutralization, overbasing, saponification, transesterification, esterification, amidification, hydrogenation, isomerization, oxidation, alkylation, acylation, sulfurization, sulfonation, rearrangement, reduction, fermentation, pyrolysis, hydrolysis, liquefaction, anaerobic digestion, hydrothermal processing, gasification or a combination of two or more thereof. Examples of natural derivatives thereof may include carboxylic acids, gums, phospholipids, soapstock, acidulated soapstock, distillate or distillate sludge, fatty acids, fatty acid esters, as well as hydroxy substituted variations thereof, including unsaturated polyol esters. In some embodiments, the natural oil derivative may comprise an unsaturated carboxylic acid having from about 5 to about 30 carbon atoms, having one or more carbon-carbon double bonds in the hydrocarbon (alkene) chain. The natural oil derivative may also comprise an unsaturated fatty acid alkyl (e.g., methyl) ester derived from a glyceride of natural oil. For example, the natural oil derivative may be a fatty acid methyl ester (“FAME”) derived from the glyceride of the natural oil. In some embodiments, a feedstock includes canola or soybean oil, as a non-limiting example, refined, bleached, and deodorized soybean oil (i.e., RBD soybean oil).

The term “free hydrocarbon” refers to any one or combination of unsaturated or saturated straight, branched, or cyclic hydrocarbons in the C₂ to C₂₄ range.

The term “metathesis monomer” refers to a single entity that is the product of a metathesis reaction which comprises a molecule of a compound with one or more carbon-carbon double bonds which has undergone an alkylidene unit interchange via one or more of the carbon-carbon double bonds either within the same molecule (intramolecular metathesis) and/or with a molecule of another compound containing one or more carbon-carbon double bonds such as an olefin (intermolecular metathesis).

The term “metathesis dimer” refers to the product of a metathesis reaction wherein two reactant compounds, which can be the same or different and each with one or more carbon-carbon double bonds, are bonded together via one or more of the carbon-carbon double bonds in each of the reactant compounds as a result of the metathesis reaction.

The term “metathesis trimer” refers to the product of one or more metathesis reactions wherein three molecules of two or more reactant compounds, which can be the same or different and each with one or more carbon-carbon double bonds, are bonded together via one or more of the carbon-carbon double bonds in each of the reactant compounds as a result of the one or more metathesis reactions, the trimer containing three bonded groups derived from the reactant compounds.

The term “metathesis tetramer” refers to the product of one or more metathesis reactions wherein four molecules of two or more reactant compounds, which can be the same or different and each with one or more carbon-carbon double bonds, are bonded together via one or more of the carbon-carbon double bonds in each of the reactant compounds as a result of the one or more metathesis reactions, the tetramer containing four bonded groups derived from the reactant compounds.

The term “metathesis pentamer” refers to the product of one or more metathesis reactions wherein five molecules of two or more reactant compounds, which can be the same or different and each with one or more carbon-carbon double bonds, are bonded together via one or more of the carbon-carbon double bonds in each of the reactant compounds as a result of the one or more metathesis reactions, the pentamer containing five bonded groups derived from the reactant compounds.

The term “metathesis hexamer” refers to the product of one or more metathesis reactions wherein six molecules of two or more reactant compounds, which can be the same or different and each with one or more carbon-carbon double bonds, are bonded together via one or more of the carbon-carbon double bonds in each of the reactant compounds as a result of the one or more metathesis reactions, the hexamer containing six bonded groups derived from the reactant compounds.

The term “metathesis heptamer” refers to the product of one or more metathesis reactions wherein seven molecules of two or more reactant compounds, which can be the same or different and each with one or more carbon-carbon double bonds, are bonded together via one or more of the carbon-carbon double bonds in each of the reactant compounds as a result of the one or more metathesis reactions, the heptamer containing seven bonded groups derived from the reactant compounds.

The term “metathesis octamer” refers to the product of one or more metathesis reactions wherein eight molecules of two or more reactant compounds, which can be the same or different and each with one or more carbon-carbon double bonds, are bonded together via one or more of the carbon-carbon double bonds in each of the reactant compounds as a result of the one or more metathesis reactions, the octamer containing eight bonded groups derived from the reactant compounds.

The term “metathesis nonamer” refers to the product of one or more metathesis reactions wherein nine molecules of two or more reactant compounds, which can be the same or different and each with one or more carbon-carbon double bonds, are bonded together via one or more of the carbon-carbon double bonds in each of the reactant compounds as a result of the one or more metathesis reactions, the nonamer containing nine bonded groups derived from the reactant compounds.

The term “metathesis decamer” refers to the product of one or more metathesis reactions wherein ten molecules of two or more reactant compounds, which can be the same or different and each with one or more carbon-carbon double bonds, are bonded together via one or more of the carbon-carbon double bonds in each of the reactant compounds as a result of the one or more metathesis reactions, the decamer containing ten bonded groups derived from the reactant compounds.

The term “metathesis oligomer” refers to the product of one or more metathesis reactions wherein two or more molecules (e.g., 2 to about 10, or 2 to about 4) of two or more reactant compounds, which can be the same or different and each with one or more carbon-carbon double bonds, are bonded together via one or more of the carbon-carbon double bonds in each of the reactant compounds as a result of the one or more metathesis reactions, the oligomer containing a few (e.g., 2 to about 10, or 2 to about 4) bonded groups derived from the reactant compounds. In some embodiments, the term “metathesis oligomer” may include metathesis reactions wherein greater than ten molecules of two or more reactant compounds, which can be the same or different and each with one or more carbon-carbon double bonds, are bonded together via one or more of the carbon-carbon double bonds in each of the reactant compounds as a result of the one or more metathesis reactions, the oligomer containing greater than ten bonded groups derived from the reactant compounds.

As used herein, the terms “metathesize” and “metathesizing” may refer to the reacting of an unsaturated polyol ester feedstock in the presence of a metathesis catalyst to form a metathesized unsaturated polyol ester product comprising a new olefinic compound and/or esters. Metathesizing may refer to cross-metathesis (a.k.a. co-metathesis), self-metathesis, ring-opening metathesis, ring-opening metathesis polymerizations (“ROMP”), ring-closing metathesis (“RCM”), and acyclic diene metathesis (“ADMET”). As a non-limiting example, metathesizing may refer to reacting two triglycerides present in a natural feedstock (self-metathesis) in the presence of a metathesis catalyst, wherein each triglyceride has an unsaturated carbon-carbon double bond, thereby forming an oligomer having a new mixture of olefins and esters that may comprise one or more of: metathesis monomers, metathesis dimers, metathesis trimers, metathesis tetramers, metathesis pentamers, and higher order metathesis oligomers (e.g., metathesis hexamers, metathesis, metathesis heptamers, metathesis octamers, metathesis nonamers, metathesis decamers, and higher than metathesis decamers and above).

The term “Oligomer Index” is defined in Section B of the Test Methods section below.

As used herein, the term “polyol” means an organic material comprising at least two hydroxy moieties.

As used herein, the term “cleaning and/or treatment composition” is a subset of consumer products that includes beauty care products. Such products include, but are not limited to, products for treating hair (human, dog, and/or cat), including, bleaching, coloring, dyeing, conditioning, shampooing, styling; deodorants and antiperspirants; personal cleansing; cosmetics; skin care including application of creams, lotions, and other topically applied products for consumer use.

As used herein, the articles including “a” and “an” when used in a claim, are understood to mean one or more of what is claimed or described.

As used herein, the terms “include”, “includes” and “including” are meant to be non-limiting.

Unless otherwise noted, all component or composition levels are in reference to the active portion of that component or composition, and are exclusive of impurities, for example, residual solvents or by-products, which may be present in commercially available sources of such components or compositions.

All percentages and ratios are calculated by weight unless otherwise indicated. All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

### Compositions and Methods of Use

TABLE 1
Compositions

| Comp. | | |
|---|---|---|
| No. | Composition | |
| 1 | A composition comprising, | |
| | a) | a metathesized unsaturated polyol ester, said metathesized |
| | | unsaturated polyol ester having one or more of the following |
| | | properties: |
| | | (i) a free hydrocarbon content, based on total weight of |
| | | metathesized unsaturated polyol ester, of from about 0% to |
| | | about 5%; |
| | | (ii) a weight average molecular weight of from about |
| | | 5,000 Daltons to about 50,000 Daltons, from about 5,500 |
| | | Daltons to about 50,000 Daltons, from about 5,500 Daltons to |
| | | about 40,000 Daltons, or from about 6,000 Daltons to about |
| | | 30,000 Daltons; |
| | | (iii) an iodine value of from about 30 to about 200, from |
| | | about 30 to about 150, from about 30 to about 120, or from |
| | | about 50 to about 110; |
| | b) | a gel matrix phase comprising: (i) from about 0.1% to about |
| | | 20% of one or more high melting point fatty compounds, by |
| | | weight of said hair care composition; (ii) from about 0.1% to |
| | | about 10% of a cationic surfactant system, by weight of said |
| | | hair care composition; and (iii) at least about 20% of an |
| | | aqueous carrier, by weight of said hair care composition. |

| | |
|---|---|
| 2 | In one aspect of said composition 1 of Table 1, said metathesized unsaturated |
| | polyol ester has the free hydrocarbon content property from a)(i) above. |
| 3 | In one aspect of said composition 1 of Table 1, said metathesized unsaturated |
| | polyol ester has the weight average molecular weight property from a)(ii) |
| | above. |
| 4 | In one aspect of said composition 1 of Table 1, said metathesized unsaturated |
| | polyol ester has the iodine value property from a)(iii) above. |
| 5 | In one aspect of said composition 1 of Table 1, said metathesized unsaturated |
| | polyol ester has the property from a)(i) and from a)(ii) above. |
| 6 | In one aspect of said composition 1 of Table 1, said metathesized unsaturated |
| | polyol ester has the properties from a)(i) and from a)(iii) above. |
| 7 | In one aspect of said composition 1 of Table 1, said metathesized unsaturated |
| | polyol ester has the properties from a)(ii) and from a)(iii) above. |
| 8 | In one aspect of said composition 1 of Table 1, said metathesized unsaturated |
| | polyol ester has the properties from a)(i), a)(ii) and from a)(iii) above. |
| 9 | In one aspect of compositions 1, 2, 3, 4, 5, 6, 7, and 8 of Table 1, said |
| | metathesized unsaturated polyol ester has a free hydrocarbon content, based |
| | on total weight of metathesized unsaturated polyol ester, of from about 0% to |
| | about 5%, from about 0.1% to about 5%, from about 0.1% to about 4%, or |
| | from about 0.1 to about 3%. |
| 10 | In one aspect of Table 1 Compositions 1, 2, 3, 4, 5, 6, 7, 8, and 9 the |
| | metathesized unsaturated polyol ester is metathesized at least once. |
| 11 | In one aspect of said composition 1, 2, 3, 4, 5, 6, 7, 8, 9, and 10 of Table 1, |
| | said metathesized unsaturated polyol ester has an oligomer index from |
| | greater than 0 to 1, from 0.001 to 1, 0.01 to 1, or from 0.05 to 1. |
| 12 | In one aspect, of compositions 1, 2, 3, 4, 5, 6, 7, 8, 9, 10 and 11 of Table 1, |
| | said composition comprises, based on total composition weight, from about |
| | 0.05% to about 30%, from about 0.1% to about 15%, from about 0.25% to |
| | about 10%, or from about 0.5% to about 5% of said metathesized unsaturated |
| | polyol ester. |

TABLE 2
Compositions

| Comp. | | |
|---|---|---|
| No. | Composition | |
| 1 | A composition comprising: | |
| | a) | a metathesized unsaturated polyol ester, said metathesized |
| | | unsaturated polyol ester having a weight average molecular |
| | | weight of from about 2,000 Daltons to about 50,000 |
| | | Daltons, from about 2,500 Daltons to about 50,000 Daltons, |
| | | from about 3,000 Daltons to about 40,000 Daltons, from |
| | | about 4,000 Daltons to about 30,000 Daltons, from about |
| | | 5,000 Daltons to about 30,000 Daltons; and one or more of |
| | | the following properties: |
| | | (i) a free hydrocarbon content, based on total weight of |
| | | metathesized unsaturated polyol ester, of from about 0% to |
| | | about 5%, from about 0.1% to about 5%, from about 0.1% |
| | | to about 4%, or from about 0.1 to about 3%; |
| | | (ii) an iodine value of from about 8 to about 200, from |
| | | about 10 to about 200, from about 20 to about 150, from |
| | | about 30 to about 120; and |
| | b) | a gel matrix phase comprising: (i) from about 0.1% to about |
| | | 20% of one or more high melting point fatty compounds, by |
| | | weight of said hair care composition; (ii) from about 0.1% to |
| | | about 10% of a cationic surfactant system, by weight of said |
| | | hair care composition; and (iii) at least about 20% of an |
| | | aqueous carrier, by weight of said hair care composition. |

| | |
|---|---|
| 2 | In one aspect of said composition 1 of Table 2, said metathesized |
| | unsaturated polyol ester has the free hydrocarbon content property from |
| | a)(i) above. |
| 3 | In one aspect of said composition 1 of Table 2, said metathesized |
| | unsaturated polyol ester has the iodine value property from a)(ii) above. |
| 4 | In one aspect of said composition 1 of Table 2, said metathesized unsaturated |
| | polyol ester has the property from a)(i) and from a)(ii) above. |
| 5 | In one aspect of Table 2, compositions 1, 2, 3 and 4, said metathesized |
| | unsaturated polyol ester has an oligomer index from greater than 0 to 1, |
| | from 0.001 to 1, 0.01 to 1, or from 0.05 to 1. |
| 6 | In one aspect of Table 2, for compositions 1, 2, 3, 4, and 5, said |
| | metathesized unsaturated polyol ester is metathesized at least once. |
| 7 | In one aspect of Table 2, for compositions 1, 2, 3, 4, 5, and 6, said |
| | composition comprises, based on total composition weight, from about |
| | 0.05% to about 30%, from about 0.1% to about 15%, from about 0.25% to |
| | about 10%, or from about 0.5% to about 5% of said metathesized unsaturated |
| | polyol ester. |

In one aspect, Table 1 Compositions 1, 2, 3, 4, 5, 6, 7, 8, 9, 10, 11 and 12; and Table 2 Compositions 1, 2, 3, 4, 5, 6, and 7 comprise one or more of the following:
a) as cationic surfactants, mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt and combinations thereof;
b) a fatty alcohol having from about 14 to about 30 carbon atoms, from about 16 to about 22 carbon atoms;
c) from about 20 wt % to about 95 wt %, or from about 60 wt % to about 85 wt %, aqueous carrier;
d) from about 0.01% to about 10%, from about 0.1% to about 8%, or from about 0.2% to about 4% of one or more additional conditioning agents;
e) a benefit agent comprising a material selected from the group consisting of anti-dandruff agents, vitamins, lipid soluble vitamins, chelants, perfumes, brighteners, enzymes, sensates, attractants, anti-bacterial agents, dyes, pigments, bleaches, and mixtures thereof; and
f) mixture thereof.

In one aspect, for Table 1 Compositions 1, 2, 3, 4, 5, 6, 7, 8, 9, 10, 11, and 12; and Table 2 Compositions 1, 2, 3, 4, 5, 6, and 7, the metathesized unsaturated polyol ester is derived from a natural polyol ester and/or a synthetic polyol ester, in one aspect, said natural polyol ester is selected from the group consisting of a vegetable oil, an animal fat, an algae oil and mixtures thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, in one aspect, sucrose, and mixtures thereof.

In one aspect, for Table 1 Compositions 1, 2, 3, 4, 5, 6, 7, 8, 9, 10, 11, and 12; and Table 2 Compositions 1, 2, 3, 4, 5, 6, and 7, the metathesized unsaturated polyol ester is selected from the group consisting of metathesized Abyssinian oil, metathesized Almond oil, metathesized Apricot oil, metathesized Apricot Kernel oil, metathesized Argan oil, metathesized Avocado oil, metathesized Babassu oil, metathesized Baobab oil, metathesized Black Cumin oil, metathesized Black Currant oil, metathesized Borage oil, metathesized Camelina oil, metathesized Carinata oil, metathesized Canola oil, metathesized Castor oil, metathesized Cherry Kernel oil, metathesized Coconut oil, metathesized Corn oil, metathesized Cottonseed oil, metathesized Echium oil, metathesized Evening Primrose oil, metathesized Flax Seed oil, metathesized Grape Seed oil, metathesized Grapefruit Seed oil, metathesized Hazelnut oil, metathesized Hemp Seed oil, metathesized Jatropha oil, metathesized Jojoba oil, metathesized Kukui Nut oil, metathesized Linseed oil, metathesized Macadamia Nut oil, metathesized Meadowfoam Seed oil, metathesized Moringa oil, metathesized Neem oil, metathesized Olive oil, metathesized Palm oil, metathesized Palm Kernel oil, metathesized Peach Kernel oil, metathesized Peanut oil, metathesized Pecan oil, metathesized Pennycress oil, metathesized Perilla Seed oil, metathesized Pistachio oil, metathesized Pomegranate Seed oil, metathesized Pongamia oil, metathesized Pumpkin Seed oil, metathesized Raspberry oil, metathesized Red Palm Olein, metathesized Rice Bran oil, metathesized Rosehip oil, metathesized Safflower oil, metathesized Seabuckthorn Fruit oil, metathesized Sesame Seed oil, metathesized Shea Olein, metathesized Sunflower oil, metathesized Soybean oil, metathesized Tonka Bean oil, metathesized Tung oil, metathesized Walnut oil, metathesized Wheat Germ oil, metathesized High Oleoyl Soybean oil, metathesized High Oleoyl Sunflower oil, metathesized High Oleoyl Safflower oil, metathesized High Erucic Acid Rapeseed oil, and mixtures thereof.

### Methods of Making Compositions

The compositions of the present invention can be formulated into any suitable form and prepared by any process chosen by the formulator, non-limiting examples of which are described in U.S. Pat. No. 5,879,584 and U.S. patent application Ser. No. 12/491,478, which are incorporated herein by reference. For example, the metathesized unsaturated polyol esters can be combined directly with the composition's other ingredients without pre-emulsification and/or pre-mixing to form the finished products. Alternatively, the metathesized unsaturated polyol esters can be combined with surfactants or emulsifiers, solvents, suitable adjuncts, and/or any other suitable ingredients to prepare emulsions prior to compounding the finished products. In some embodiments, the metathesized polyol esters can be added to the composition separately from the gel matrix. In such embodiments, where there is a discrete phase comprising the metathesized polyol esters, the discrete phase can optionally have an average particle size in the hair care composition of from about 0.5 μm to about 20 μm. In other embodiments, the metathesized polyol esters can be added to the gel matrix first and then this gel matrix is combined with other components of the composition.

Suitable equipment for use in the processes disclosed herein may include continuous stirred tank reactors, homogenizers, turbine agitators, recirculating pumps, paddle mixers, plough shear mixers, ribbon blenders, vertical axis granulators and drum mixers, both in batch and, where available, in continuous process configurations, spray dryers, and extruders. Such equipment can be obtained from Lodige GmbH (Paderborn, Germany), Littleford Day, Inc. (Florence, Ky., U.S.A.), Forberg AS (Larvik, Norway), Glatt Ingenieurtechnik GmbH (Weimar, Germany), Niro (Soeborg, Denmark), Hosokawa Bepex Corp. (Minneapolis, Minn., U.S.A.), Arde Barinco (New Jersey, U.S.A.).

### A. Metathesized Unsaturated Polyol Ester

The hair care composition comprises, based on total composition weight, from about 0.05% to about 30%, from about 0.1% to about 15%, from about 0.25% to about 10%, or from about 0.5% to about 5%, of the metathesized unsaturated polyol ester.

Exemplary metathesized unsaturated polyol esters and their starting materials are set forth in U.S. Patent Applications U.S. 2009/0220443 A1, U.S. 2013/0344012 A1 and US 2014/0357714 A1, which are incorporated herein by reference. A metathesized unsaturated polyol ester refers to the product obtained when one or more unsaturated polyol ester ingredient(s) are subjected to a metathesis reaction. Metathesis is a catalytic reaction that involves the interchange of alkylidene units among compounds containing one or more double bonds (i.e., olefinic compounds) via the formation and cleavage of the carbon-carbon double bonds. Metathesis may occur between two of the same molecules (often referred to as self-metathesis) and/or it may occur between two different molecules (often referred to as cross-metathesis). Self-metathesis may be represented schematically as shown in Equation I.

R¹—CH═CH—R²+R¹—CH═CH—R² [symbol] R¹—CH═CH—R¹+R²—CH═CH—R² (I)

where R¹ and R² are organic groups.

R¹—CH═CH—R²+R³—CH═CH—R⁴ [symbol] R¹—CH═CH—R³+R¹—CH═CH—R⁴+R²—CH═CH—R³+R²—CH═CH—R⁴+R¹—CH═CH—R¹+R²—CH═CH—R²+R³—CH═CH—R³+R⁴—CH═CH—R⁴ (II)

Cross-metathesis may be represented schematically as shown in Equation II.

where R¹, R², R³, and R⁴ are organic groups.

When a polyol ester comprises molecules having more than one carbon-carbon double bond, self-metathesis may result in oligomerization or polymerization of the unsaturates in the starting material. For example, Equation C depicts metathesis oligomerization of a representative species (e.g., a polyol ester) having more than one carbon-carbon double bond. In Equation C, the self-metathesis reaction results in the formation of metathesis dimers, metathesis trimers, and metathesis tetramers. Although not shown, higher order oligomers such as metathesis pentamers, hexamers, heptamers, octamers, nonamers, decamers, and higher than decamers, and mixtures of two or more thereof, may also be formed. The number of metathesis repeating units or groups in the metathesized natural oil may range from 1 to about 100, or from 2 to about 50, or from 2 to about 30, or from 2 to about 10, or from 2 to about 4. The molecular weight of the metathesis dimer may be greater than the molecular weight of the unsaturated polyol ester from which the dimer is formed. Each of the bonded polyol ester molecules may be referred to as a “repeating unit or group.” Typically, a metathesis trimer may be formed by the cross-metathesis of a metathesis dimer with an unsaturated polyol ester. Typically, a metathesis tetramer may be formed by the cross-metathesis of a metathesis trimer with an unsaturated polyol ester or formed by the cross-metathesis of two metathesis dimers.

Equation C

R¹—HC═CH—R²—HC═CH—R³+R¹—HC═CH—R²—HC═CH—R³ [symbol] R¹—HC═CH—R²—HC═CH—R²—HC═CH—R³+(other products) (metathesis dimer)

R¹—R²—HC═CH—R²—HC═CH—R³+R¹—HC═CH—R²—HC═CH—R³ [symbol] R¹—HC═CH—R²—HC═CH—R²—HC═CH—R²—HC═CH—R³+(other products) (metathesis trimer)

R¹—HC═CH—R²—HC═CH—R²—HC═CH—R²—HC═CH—R³+R¹—HC═CH—R²—HC═CH—R³ [symbol] R¹—HC═CH—R²—HC═CH—R²—HC═CH—R²—HC═CH—R²—HC═CH—R³+(other products) (metathesis tetramer)

where R¹, R², and R³ are organic groups.

As a starting material, metathesized unsaturated polyol esters are prepared from one or more unsaturated polyol esters. As used herein, the term “unsaturated polyol ester” refers to a compound having two or more hydroxyl groups wherein at least one of the hydroxyl groups is in the form of an ester and wherein the ester has an organic group including at least one carbon-carbon double bond. In many embodiments, the unsaturated polyol ester can be represented by the general structure (I):

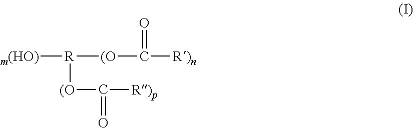

where n≧1; m≧0; p≧0; (n+m+p)≧2; R is an organic group; R′ is an organic group having at least one carbon-carbon double bond; and R″ is a saturated organic group.

In many embodiments of the invention, the unsaturated polyol ester is an unsaturated polyol ester of glycerol. Unsaturated polyol esters of glycerol have the general structure (II):

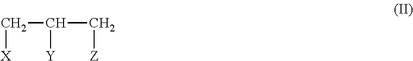

where —X, —Y, and —Z are independently selected from the group consisting of:

—OH; —(O—C(═O)—R′); and —(O—C(═O)—R″);

where —R′ is an organic group having at least one carbon-carbon double bond and —R″ is a saturated organic group.

In structure (II), at least one of —X, —Y, and —Z is —(O—C(═O)—R′).

In some embodiments, R′ is a straight or branched chain hydrocarbon having about 50 or less carbon atoms (e.g., about 36 or less carbon atoms or about 26 or less carbon atoms) and at least one carbon-carbon double bond in its chain. In some embodiments, R′ is a straight or branched chain hydrocarbon having about 6 carbon atoms or greater (e.g., about 10 carbon atoms or greater or about 12 carbon atoms or greater) and at least one carbon-carbon double bond in its chain. In some embodiments, R′ may have two or more carbon-carbon double bonds in its chain. In other embodiments, R may have three or more double bonds in its chain. In exemplary embodiments, R has 17 carbon atoms and 1 to 3 carbon-carbon double bonds in its chain. Representative examples of R′ include:

—(CH₂)₇CH═CH—(CH₂)₇—CH₃;

—(CH₂)₇CH═CH—CH₂—CH═CH—(CH₂)₄—CH₃; and

—(CH₂)₇CH═CH—CH₂—CH═CH—CH₂—CH═CH—CH₂—CH₃.

In some embodiments, R″ is a saturated straight or branched chain hydrocarbon having about 50 or less carbon atoms (e.g., about 36 or less carbon atoms or about 26 or less carbon atoms). In some embodiments, R″ is a saturated straight or branched chain hydrocarbon having about 6 carbon atoms or greater (e.g., about 10 carbon atoms or greater or about 12 carbon atoms or greater. In exemplary embodiments, R″ has 15 carbon atoms or 17 carbon atoms.

Sources of unsaturated polyol esters of glycerol include synthesized oils, natural oils (e.g., vegetable oils, algae oils, bacterial derived oils, and animal fats), combinations of these, and the like. Recycled used vegetable oils may also be used. Representative non-limiting examples of vegetable oils include Abyssinian oil, Almond oil, Apricot oil, Apricot Kernel oil, Argan oil, Avocado oil, Babassu oil, Baobab oil, Black Cumin oil, Black Currant oil, Borage oil, Camelina oil, Carinata oil, Canola oil, Castor oil, Cherry Kernel oil, Coconut oil, Corn oil, Cottonseed oil, Echium oil, Evening Primrose oil, Flax Seed oil, Grape Seed oil, Grapefruit Seed oil, Hazelnut oil, Hemp Seed oil, Jatropha oil, Jojoba oil, Kukui Nut oil, Linseed oil, Macadamia Nut oil, Meadowfoam Seed oil, Moringa oil, Neem oil, Olive oil, Palm oil, Palm Kernel oil, Peach Kernel oil, Peanut oil, Pecan oil, Pennycress oil, Perilla Seed oil, Pistachio oil, Pomegranate Seed oil, Pongamia oil, Pumpkin Seed oil, Raspberry oil, Red Palm Olein, Rice Bran oil, Rosehip oil, Safflower oil, Seabuckthorn Fruit oil, Sesame Seed oil, Shea Olein, Sunflower oil, Soybean oil, Tonka Bean oil, Tung oil, Walnut oil, Wheat Germ oil, High Oleoyl Soybean oil, High Oleoyl Sunflower oil, High Oleoyl Safflower oil, High Erucic Acid Rapeseed oil, combinations of these, and the like. Representative non-limiting examples of animal fats include lard, tallow, chicken fat, yellow grease, fish oil, emu oil, combinations of these, and the like. A representative non-limiting example of a synthesized oil includes tall oil, which is a byproduct of wood pulp manufacture. In some embodiments, the natural oil is refined, bleached, and/or deodorized.

Other examples of unsaturated polyol esters include esters such as those derived from ethylene glycol or propylene glycol, polyethylene glycol, polypropylene glycol, or poly(tetramethylene ether) glycol, esters such as those derived from pentaerythritol, dipentaerythritol, tripentaerythritol, trimethylolpropane, or neopentyl glycol, or sugar esters such as SEFOSE®. Sugar esters such as SEFOSE® include one or more types of sucrose polyesters, with up to eight ester groups that could undergo a metathesis exchange reaction. Sucrose polyesters are derived from a natural resource and therefore, the use of sucrose polyesters can result in a positive environmental impact. Sucrose polyesters are polyester materials, having multiple substitution positions around the sucrose backbone coupled with the chain length, saturation, and derivation variables of the fatty chains. Such sucrose polyesters can have an esterification (“IBAR”) of greater than about 5. In one embodiment the sucrose polyester may have an IBAR of from about 5 to about 8. In another embodiment the sucrose polyester has an IBAR of about 5-7, and in another embodiment the sucrose polyester has an IBAR of about 6. In yet another embodiment the sucrose polyester has an IBAR of about 8. As sucrose polyesters are derived from a natural resource, a distribution in the IBAR and chain length may exist. For example a sucrose polyester having an IBAR of 6, may contain a mixture of mostly IBAR of about 6, with some IBAR of about 5 and some IBAR of about 7. Additionally, such sucrose polyesters may have a saturation or iodine value (“IV”) of about 3 to about 140. In another embodiment the sucrose polyester may have an IV of about 10 to about 120. In yet another embodiment the sucrose polyester may have an IV of about 20 to 100. Further, such sucrose polyesters have a chain length of about C₁₂ to C₂₀ but are not limited to these chain lengths.

Non-limiting examples of sucrose polyesters suitable for use include SEFOSE® 1618S, SEFOSE® 1618U, SEFOSE® 1618H, Sefa Soyate IMF 40, Sefa Soyate LP426, SEFOSE® 2275, SEFOSE® C1695, SEFOSE® C18:0 95, SEFOSE® C1495, SEFOSE® 1618H B6, SEFOSE® 1618S B6, SEFOSE® 1618U B6, Sefa Cottonate, SEFOSE® C1295, Sefa C895, Sefa C1095, SEFOSE® 1618S B4.5, all available from The Procter and Gamble Co. of Cincinnati, Ohio.

Other examples of suitable polyol esters may include but not be limited to sorbitol esters, maltitol esters, sorbitan esters, maltodextrin derived esters, xylitol esters, polyglycerol esters, and other sugar derived esters.

Natural oils of the type described herein typically are composed of triglycerides of fatty acids. These fatty acids may be either saturated, monounsaturated or polyunsaturated and contain varying chain lengths ranging from C₈ to C₃₀. The most common fatty acids include saturated fatty acids such as lauric acid (dodecanoic acid), myristic acid (tetradecanoic acid), palmitic acid (hexadecanoic acid), stearic acid (octadecanoic acid), arachidic acid (eicosanoic acid), and lignoceric acid (tetracosanoic acid); unsaturated acids include such fatty acids as palmitoleic (a C₁₆ acid), and oleic acid (a C₁₈ acid); polyunsaturated acids include such fatty acids as linoleic acid (a di-unsaturated C₁₈ acid), linolenic acid (a tri-unsaturated C₁₈ acid), and arachidonic acid (a tetra-unsubstituted C₂₀ acid). The natural oils are further comprised of esters of these fatty acids in random placement onto the three sites of the trifunctional glycerine molecule. Different natural oils will have different ratios of these fatty acids, and within a given natural oil there is a range of these acids as well depending on such factors as where a vegetable or crop is grown, maturity of the vegetable or crop, the weather during the growing season, etc. Thus, it is difficult to have a specific or unique structure for any given natural oil, but rather a structure is typically based on some statistical average. For example soybean oil contains a mixture of stearic acid, oleic acid, linoleic acid, and linolenic acid in the ratio of 15:24:50:11, and an average number of double bonds of 4.4-4.7 per triglyceride. One method of quantifying the number of double bonds is the iodine value (IV) which is defined as the number of grams of iodine that will react with 100 grams of oil. Therefore for soybean oil, the average iodine value range is from 120-140. Soybean oil may comprises about 95% by weight or greater (e.g., 99% weight or greater) triglycerides of fatty acids. Major fatty acids in the polyol esters of soybean oil include saturated fatty acids, as a non-limiting example, palmitic acid (hexadecanoic acid) and stearic acid (octadecanoic acid), and unsaturated fatty acids, as a non-limiting example, oleic acid (9-octadecenoic acid), linoleic acid (9,12octadecadienoic acid), and linolenic acid (9,12,15-octadecatrienoic acid).

In an exemplary embodiment, the vegetable oil is canola oil, for example, refined, bleached, and deodorized canola oil (i.e., RBD canola oil). Canola oil is an unsaturated polyol ester of glycerol that typically comprises about 95% weight or greater (e.g., 99% weight or greater) triglycerides of fatty acids. Major fatty acids in the polyol esters of canola oil include saturated fatty acids, for example, palmitic acid (hexadecanoic acid) and stearic acid (octadecanoic acid), and unsaturated fatty acids, for example, oleic acid (9-octadecenoic acid), linoleic acid (9,12-octadecadienoic acid), and linolenic acid (9,12,15-octadecatrienoic acid). Canola oil is a highly unsaturated vegetable oil with many of the triglyceride molecules having at least two unsaturated fatty acids (i.e., a polyunsaturated triglyceride).

In exemplary embodiments, an unsaturated polyol ester is self-metathesized in the presence of a metathesis catalyst to form a metathesized composition. Typically, after metathesis has occurred, the metathesis catalyst is removed from the resulting product. One method of removing the catalyst is treatment of the metathesized product with clay. In many embodiments, the metathesized composition comprises one or more of: metathesis monomers, metathesis dimers, metathesis trimers, metathesis tetramers, metathesis pentamers, and higher order metathesis oligomers (e.g., metathesis hexamers). A metathesis dimer refers to a compound formed when two unsaturated polyol ester molecules are covalently bonded to one another by a self-metathesis reaction. In many embodiments, the molecular weight of the metathesis dimer is greater than the molecular weight of the individual unsaturated polyol ester molecules from which the dimer is formed. A metathesis trimer refers to a compound formed when three unsaturated polyol ester molecules are covalently bonded together by metathesis reactions. In many embodiments, a metathesis trimer is formed by the cross-metathesis of a metathesis dimer with an unsaturated polyol ester. A metathesis tetramer refers to a compound formed when four unsaturated polyol ester molecules are covalently bonded together by metathesis reactions. In many embodiments, a metathesis tetramer is formed by the cross-metathesis of a metathesis trimer with an unsaturated polyol ester. Metathesis tetramers may also be formed, for example, by the cross-metathesis of two metathesis dimers. Higher order metathesis products may also be formed. For example, metathesis pentamers and metathesis hexamers may also be formed. The self-metathesis reaction also results in the formation of internal olefin compounds that may be linear or cyclic. If the metathesized polyol ester is fully or partially hydrogenated, the linear and cyclic olefins would typically be fully or partially converted to the corresponding saturated linear and cyclic hydrocarbons. The linear/cyclic olefins and saturated linear/cyclic hydrocarbons may remain in the metathesized polyol ester or they may be removed or partially removed from the metathesized polyol ester using one or more known stripping techniques, including but not limited to wipe film evaporation, falling film evaporation, rotary evaporation, steam stripping, vacuum distillation, etc.

In some embodiments, the unsaturated polyol ester is partially hydrogenated before being metathesized. For example, in some embodiments, the unsaturated polyol ester is partially hydrogenated to achieve an iodine value (IV) of about 120 or less before subjecting the partially hydrogenated polyol ester to metathesis.

In some embodiments, the unsaturated polyol ester may be hydrogenated (e.g., fully or partially hydrogenated) in order to improve the stability of the oil or to modify its viscosity or other properties. Representative techniques for hydrogenating unsaturated polyol esters are known in the art and are discussed herein.

In some embodiments, the natural oil is winterized. Winterization refers to the process of: (1) removing waxes and other non-triglyceride constituents, (2) removing naturally occurring high-melting triglycerides, and (3) removing high-melting triglycerides formed during partial hydrogenation. Winterization may be accomplished by known methods including, for example, cooling the oil at a controlled rate in order to cause crystallization of the higher melting components that are to be removed from the oil. The crystallized high melting components are then removed from the oil by filtration resulting in winterized oil. Winterized soybean oil is commercially available from Cargill, Incorporated (Minneapolis, Minn.).

In other embodiments, the metathesized unsaturated polyol esters can be used as a blend with one or more fabric care benefit agents and/or fabric softening actives.

### Method of Making Metathesized Unsaturated Polyol Ester

The self-metathesis of unsaturated polyol esters is typically conducted in the presence of a catalytically effective amount of a metathesis catalyst. The term “metathesis catalyst” includes any catalyst or catalyst system that catalyzes a metathesis reaction. Any known or future-developed metathesis catalyst may be used, alone or in combination with one or more additional catalysts. Suitable homogeneous metathesis catalysts include combinations of a transition metal halide or oxo-halide (e.g., WOCl₄ or WCl₆) with an alkylating cocatalyst (e.g., Me₄Sn), or alkylidene (or carbene) complexes of transition metals, particularly Ru or W. These include first and second-generation Grubbs catalysts, Grubbs-Hoveyda catalysts, and the like. Suitable alkylidene catalysts have the general structure:

M[X¹X²L¹L²(L³)ₙ]═Cₘ═C(R¹)R²

where M is a Group 8 transition metal, L¹, L², and L³ are neutral electron donor ligands, n is 0 (such that L³ may not be present) or 1, m is 0, 1, or 2, X¹ and X² are anionic ligands, and R¹ and R² are independently selected from H, hydrocarbyl, substituted hydrocarbyl, heteroatom-containing hydrocarbyl, substituted heteroatom-containing hydrocarbyl, and functional groups. Any two or more of X¹, X², L¹, L², L³, R¹ and R² can form a cyclic group and any one of those groups can be attached to a support.

First-generation Grubbs catalysts fall into this category where m=n=0 and particular selections are made for n, X¹, X², L¹, L², L³, R¹ and R² as described in U.S. Pat. Appl. Publ. No. 2010/0145086, the teachings of which related to all metathesis catalysts are incorporated herein by reference.

Second-generation Grubbs catalysts also have the general formula described above, but L¹ is a carbene ligand where the carbene carbon is flanked by N, O, S, or P atoms, preferably by two N atoms. Usually, the carbene ligand is part of a cyclic group. Examples of suitable second-generation Grubbs catalysts also appear in the '086 publication.

In another class of suitable alkylidene catalysts, L¹ is a strongly coordinating neutral electron donor as in first- and second-generation Grubbs catalysts, and L² and L³ are weakly coordinating neutral electron donor ligands in the form of optionally substituted heterocyclic groups. Thus, L² and L³ are pyridine, pyrimidine, pyrrole, quinoline, thiophene, or the like.

In yet another class of suitable alkylidene catalysts, a pair of substituents is used to form a bi- or tridentate ligand, such as a biphosphine, dialkoxide, or alkyldiketonate. Grubbs-Hoveyda catalysts are a subset of this type of catalyst in which L² and R² are linked. Typically, a neutral oxygen or nitrogen coordinates to the metal while also being bonded to a carbon that is α-, β-, or γ- with respect to the carbene carbon to provide the bidentate ligand. Examples of suitable Grubbs-Hoveyda catalysts appear in the '086 publication.

The structures below provide just a few illustrations of suitable catalysts that may be used:

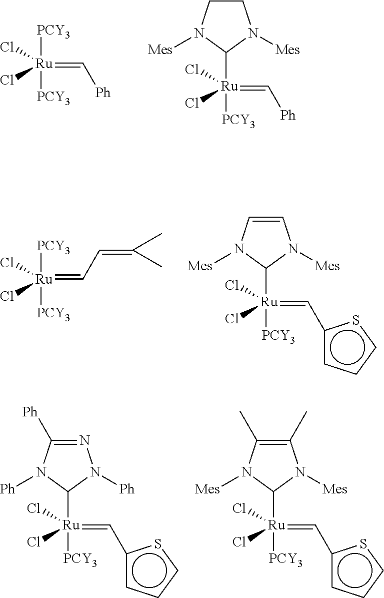

An immobilized catalyst can be used for the metathesis process. An immobilized catalyst is a system comprising a catalyst and a support, the catalyst associated with the support. Exemplary associations between the catalyst and the support may occur by way of chemical bonds or weak interactions (e.g. hydrogen bonds, donor acceptor interactions) between the catalyst, or any portions thereof, and the support or any portions thereof. Support is intended to include any material suitable to support the catalyst. Typically, immobilized catalysts are solid phase catalysts that act on liquid or gas phase reactants and products. Exemplary supports are polymers, silica or alumina. Such an immobilized catalyst may be used in a flow process. An immobilized catalyst can simplify purification of products and recovery of the catalyst so that recycling the catalyst may be more convenient.

In certain embodiments, prior to the metathesis reaction, the unsaturated polyol ester feedstock may be treated to render the natural oil more suitable for the subsequent metathesis reaction. In one embodiment, the treatment of the unsaturated polyol ester involves the removal of catalyst poisons, such as peroxides, which may potentially diminish the activity of the metathesis catalyst. Non-limiting examples of unsaturated polyol ester feedstock treatment methods to diminish catalyst poisons include those described in PCT/US2008/09604, PCT/US2008/09635, and U.S. patent application Ser. Nos. 12/672,651 and 12/672,652, herein incorporated by reference in their entireties. In certain embodiments, the unsaturated polyol ester feedstock is thermally treated by heating the feedstock to a temperature greater than 100° C. in the absence of oxygen and held at the temperature for a time sufficient to diminish catalyst poisons in the feedstock. In other embodiments, the temperature is between approximately 100° C. and 300° C., between approximately 120° C. and 250° C., between approximately 150° C. and 210° C., or approximately between 190 and 200° C. In one embodiment, the absence of oxygen is achieved by sparging the unsaturated polyol ester feedstock with nitrogen, wherein the nitrogen gas is pumped into the feedstock treatment vessel at a pressure of approximately 10 atm (150 psig).

In certain embodiments, the unsaturated polyol ester feedstock is chemically treated under conditions sufficient to diminish the catalyst poisons in the feedstock through a chemical reaction of the catalyst poisons. In certain embodiments, the feedstock is treated with a reducing agent or a cation-inorganic base composition. Non-limiting examples of reducing agents include bisulfate, borohydride, phosphine, thiosulfate, and combinations thereof.

In certain embodiments, the unsaturated polyol ester feedstock is treated with an adsorbent to remove catalyst poisons. In one embodiment, the feedstock is treated with a combination of thermal and adsorbent methods. In another embodiment, the feedstock is treated with a combination of chemical and adsorbent methods. In another embodiment, the treatment involves a partial hydrogenation treatment to modify the unsaturated polyol ester feedstocks reactivity with the metathesis catalyst. Additional non-limiting examples of feedstock treatment are also described below when discussing the various metathesis catalysts.

In certain embodiments, a ligand may be added to the metathesis reaction mixture. In many embodiments using a ligand, the ligand is selected to be a molecule that stabilizes the catalyst, and may thus provide an increased turnover number for the catalyst. In some cases the ligand can alter reaction selectivity and product distribution. Examples of ligands that can be used include Lewis base ligands, such as, without limitation, trialkylphosphines, for example tricyclohexylphosphine and tributyl phosphine; triarylphosphines, such as triphenylphosphine; diarylalkylphosphines, such as, diphenylcyclohexylphosphine; pyridines, such as 2,6-dimethylpyridine, 2,4,6-trimethylpyridine; as well as other Lewis basic ligands, such as phosphine oxides and phosphinites. Additives may also be present during metathesis that increase catalyst lifetime.

Any useful amount of the selected metathesis catalyst can be used in the process. For example, the molar ratio of the unsaturated polyol ester to catalyst may range from about 5:1 to about 10,000,000:1 or from about 50:1 to 500,000:1. In some embodiments, an amount of about 1 to about 10 ppm, or about 2 ppm to about 5 ppm, of the metathesis catalyst per double bond of the starting composition (i.e., on a mole/mole basis) is used.

In some embodiments, the metathesis reaction is catalyzed by a system containing both a transition and a non-transition metal component. The most active and largest number of catalyst systems are derived from Group VI A transition metals, for example, tungsten and molybdenum.

Multiple, sequential metathesis reaction steps may be employed. For example, the metathesized unsaturated polyol ester product may be made by reacting an unsaturated polyol ester in the presence of a metathesis catalyst to form a first metathesized unsaturated polyol ester product. The first metathesized unsaturated polyol ester product may then be reacted in a self-metathesis reaction to form another metathesized unsaturated polyol ester product. Alternatively, the first metathesized unsaturated polyol ester product may be reacted in a cross-metathesis reaction with an unsaturated polyol ester to form another metathesized unsaturated polyol ester product. Also in the alternative, the transesterified products, the olefins and/or esters may be further metathesized in the presence of a metathesis catalyst. Such multiple and/or sequential metathesis reactions can be performed as many times as needed, and at least one or more times, depending on the processing/compositional requirements as understood by a person skilled in the art. As used herein, a “metathesized unsaturated polyol ester product” may include products that have been once metathesized and/or multiply metathesized. These procedures may be used to form metathesis dimers, metathesis trimers, metathesis tetramers, metathesis pentamers, and higher order metathesis oligomers (e.g., metathesis hexamers, metathesis heptamers, metathesis octamers, metathesis nonamers, metathesis decamers, and higher than metathesis decamers). These procedures can be repeated as many times as desired (for example, from 2 to about 50 times, or from 2 to about 30 times, or from 2 to about 10 times, or from 2 to about 5 times, or from 2 to about 4 times, or 2 or 3 times) to provide the desired metathesis oligomer or polymer which may comprise, for example, from 2 to about 100 bonded groups, or from 2 to about 50, or from 2 to about 30, or from 2 to about 10, or from 2 to about 8, or from 2 to about 6 bonded groups, or from 2 to about 4 bonded groups, or from 2 to about 3 bonded groups. In certain embodiments, it may be desirable to use the metathesized unsaturated polyol ester products produced by cross metathesis of an unsaturated polyol ester, or blend of unsaturated polyol esters, with a C2-C100 olefin, as the reactant in a self-metathesis reaction to produce another metathesized unsaturated polyol ester product. Alternatively, metathesized products produced by cross metathesis of an unsaturated polyol ester, or blend of unsaturated polyol esters, with a C2-C100 olefin can be combined with an unsaturated polyol ester, or blend of unsaturated polyol esters, and further metathesized to produce another metathesized unsaturated polyol ester product.

The metathesis process can be conducted under any conditions adequate to produce the desired metathesis products. For example, stoichiometry, atmosphere, solvent, temperature, and pressure can be selected by one skilled in the art to produce a desired product and to minimize undesirable byproducts. The metathesis process may be conducted under an inert atmosphere. Similarly, if a reagent is supplied as a gas, an inert gaseous diluent can be used. The inert atmosphere or inert gaseous diluent typically is an inert gas, meaning that the gas does not interact with the metathesis catalyst to substantially impede catalysis. For example, particular inert gases are selected from the group consisting of helium, neon, argon, nitrogen, individually or in combinations thereof.

In certain embodiments, the metathesis catalyst is dissolved in a solvent prior to conducting the metathesis reaction. In certain embodiments, the solvent chosen may be selected to be substantially inert with respect to the metathesis catalyst. For example, substantially inert solvents include, without limitation, aromatic hydrocarbons, such as benzene, toluene, xylenes, etc.; halogenated aromatic hydrocarbons, such as chlorobenzene and dichlorobenzene; aliphatic solvents, including pentane, hexane, heptane, cyclohexane, etc.; and chlorinated alkanes, such as dichloromethane, chloroform, dichloroethane, etc. In one particular embodiment, the solvent comprises toluene. The metathesis reaction temperature may be a rate-controlling variable where the temperature is selected to provide a desired product at an acceptable rate. In certain embodiments, the metathesis reaction temperature is greater than about −40° C., greater than about −20° C., greater than about 0° C., or greater than about 10° C. In certain embodiments, the metathesis reaction temperature is less than about 150° C., or less than about 120° C. In one embodiment, the metathesis reaction temperature is between about 10° C. and about 120° C.

The metathesis reaction can be run under any desired pressure. Typically, it will be desirable to maintain a total pressure that is high enough to keep the cross-metathesis reagent in solution. Therefore, as the molecular weight of the cross-metathesis reagent increases, the lower pressure range typically decreases since the boiling point of the cross-metathesis reagent increases. The total pressure may be selected to be greater than about 0.1 atm (10 kPa), in some embodiments greater than about 0.3 atm (30 kPa), or greater than about 1 atm (100 kPa). Typically, the reaction pressure is no more than about 70 atm (7000 kPa), in some embodiments no more than about 30 atm (3000 kPa). A non-limiting exemplary pressure range for the metathesis reaction is from about 1 atm (100 kPa) to about 30 atm (3000 kPa). In certain embodiments it may be desirable to run the metathesis reactions under an atmosphere of reduced pressure. Conditions of reduced pressure or vacuum may be used to remove olefins as they are generated in a metathesis reaction, thereby driving the metathesis equilibrium towards the formation of less volatile products. In the case of a self-metathesis of a natural oil, reduced pressure can be used to remove C₁₂ or lighter olefins including, but not limited to, hexene, nonene, and dodecene, as well as byproducts including, but not limited to cyclohexa-diene and benzene as the metathesis reaction proceeds. The removal of these species can be used as a means to drive the reaction towards the formation of diester groups and cross linked triglycerides.

Hydrogenation:

In some embodiments, the unsaturated polyol ester is partially hydrogenated before it is subjected to the metathesis reaction. Partial hydrogenation of the unsaturated polyol ester reduces the number of double bonds that are available for in the subsequent metathesis reaction. In some embodiments, the unsaturated polyol ester is metathesized to form a metathesized unsaturated polyol ester, and the metathesized unsaturated polyol ester is then hydrogenated (e.g., partially or fully hydrogenated) to form a hydrogenated metathesized unsaturated polyol ester.

Hydrogenation may be conducted according to any known method for hydrogenating double bond-containing compounds such as vegetable oils. In some embodiments, the unsaturated polyol ester or metathesized unsaturated polyol ester is hydrogenated in the presence of a nickel catalyst that has been chemically reduced with hydrogen to an active state. Commercial examples of supported nickel hydrogenation catalysts include those available under the trade designations “NYSOFACT”, “NYSOSEL”, and “NI 5248 D” (from Englehard Corporation, Iselin, N.H.). Additional supported nickel hydrogenation catalysts include those commercially available under the trade designations “PRICAT 9910”, “PRICAT 9920”, “PRICAT 9908”, “PRICAT 9936” (from Johnson Matthey Catalysts, Ward Hill, Mass.).

In some embodiments, the hydrogenation catalyst comprising, for example, nickel, copper, palladium, platinum, molybdenum, iron, ruthenium, osmium, rhodium, or iridium. Combinations of metals may also be used. Useful catalyst may be heterogeneous or homogeneous. In some embodiments, the catalysts are supported nickel or sponge nickel type catalysts.

In some embodiments, the hydrogenation catalyst comprises nickel that has been chemically reduced with hydrogen to an active state (i.e., reduced nickel) provided on a support. In some embodiments, the support comprises porous silica (e.g., kieselguhr, infusorial, diatomaceous, or siliceous earth) or alumina. The catalysts are characterized by a high nickel surface area per gram of nickel.

In some embodiments, the particles of supported nickel catalyst are dispersed in a protective medium comprising hardened triacylglyceride, edible oil, or tallow. In an exemplary embodiment, the supported nickel catalyst is dispersed in the protective medium at a level of about 22 wt. % nickel.

Hydrogenation may be carried out in a batch or in a continuous process and may be partial hydrogenation or complete hydrogenation. In a representative batch process, a vacuum is pulled on the headspace of a stirred reaction vessel and the reaction vessel is charged with the material to be hydrogenated (e.g., RBD soybean oil or metathesized RBD soybean oil). The material is then heated to a desired temperature. Typically, the temperature ranges from about 50° C. to 350° C., for example, about 100° C. to 300° C. or about 150° C. to 250° C. The desired temperature may vary, for example, with hydrogen gas pressure. Typically, a higher gas pressure will require a lower temperature. In a separate container, the hydrogenation catalyst is weighed into a mixing vessel and is slurried in a small amount of the material to be hydrogenated (e.g., RBD soybean oil or metathesized RBD soybean oil). When the material to be hydrogenated reaches the desired temperature, the slurry of hydrogenation catalyst is added to the reaction vessel. Hydrogen gas is then pumped into the reaction vessel to achieve a desired pressure of H2 gas. Typically, the H2 gas pressure ranges from about 15 to 3000 psig, for example, about 15 psig to 90 psig. As the gas pressure increases, more specialized high-pressure processing equipment may be required. Under these conditions the hydrogenation reaction begins and the temperature is allowed to increase to the desired hydrogenation temperature (e.g., about 120° C. to 200° C.) where it is maintained by cooling the reaction mass, for example, with cooling coils. When the desired degree of hydrogenation is reached, the reaction mass is cooled to the desired filtration temperature.

The amount of hydrogenation catalysts is typically selected in view of a number of factors including, for example, the type of hydrogenation catalyst used, the amount of hydrogenation catalyst used, the degree of unsaturation in the material to be hydrogenated, the desired rate of hydrogenation, the desired degree of hydrogenation (e.g., as measure by iodine value (IV)), the purity of the reagent, and the H2 gas pressure. In some embodiments, the hydrogenation catalyst is used in an amount of about 10 wt. % or less, for example, about 5 wt. % or less or about 1 wt. % or less.

After hydrogenation, the hydrogenation catalyst may be removed from the hydrogenated product using known techniques, for example, by filtration. In some embodiments, the hydrogenation catalyst is removed using a plate and frame filter such as those commercially available from Sparkler Filters, Inc., Conroe Tex. In some embodiments, the filtration is performed with the assistance of pressure or a vacuum. In order to improve filtering performance, a filter aid may be used. A filter aid may be added to the metathesized product directly or it may be applied to the filter. Representative examples of filtering aids include diatomaceous earth, silica, alumina, and carbon. Typically, the filtering aid is used in an amount of about 10 wt. % or less, for example, about 5 wt. % or less or about 1 wt. % or less. Other filtering techniques and filtering aids may also be employed to remove the used hydrogenation catalyst. In other embodiments the hydrogenation catalyst is removed using centrifugation followed by decantation of the product.

### B. Cationic Surfactant System

The composition of the present invention comprises a cationic surfactant system. The cationic surfactant system can be one cationic surfactant or a mixture of two or more cationic surfactants. Preferably, the cationic surfactant system is selected from: mono-long alkyl quaternized ammonium salt; a combination of mono-long alkyl quaternized ammonium salt and di-long alkyl quaternized ammonium salt; mono-long alkyl amidoamine salt; a combination of mono-long alkyl amidoamine salt and di-long alkyl quaternized ammonium salt, a combination of mono-long alkyl amindoamine salt and mono-long alkyl quaternized ammonium salt.

The cationic surfactant system is included in the composition at a level by weight of from about 0.1% to about 10%, preferably from about 0.5% to about 8%, more preferably from about 0.8% to about 5%, still more preferably from about 1.0% to about 4%.

Mono-Long Alkyl Quaternized Ammonium Salt

The monoalkyl quaternized ammonium salt cationic surfactants useful herein are those having one long alkyl chain which has from 12 to 30 carbon atoms, preferably from 16 to 24 carbon atoms, more preferably C18-22 alkyl group. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Mono-long alkyl quaternized ammonium salts useful herein are those having the formula (I):

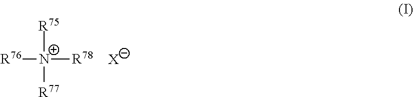

wherein one of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium salt; stearyl trimethyl ammonium salt; cetyl trimethyl ammonium salt; and hydrogenated tallow alkyl trimethyl ammonium salt.

Mono-Long Alkyl Amidoamine Salt

Mono-long alkyl amines are also suitable as cationic surfactants. Primary, secondary, and tertiary fatty amines are useful. Particularly useful are tertiary amido amines having an alkyl group of from about 12 to about 22 carbons. Exemplary tertiary amido amines include: stearamidopropyldimethylamine, stearamidopropyldiethylamine, stearamidoethyldiethylamine, stearamidoethyldimethylamine, palmitamidopropyldimethylamine, palmitamidopropyldiethylamine, palmitamidoethyldiethylamine, palmitamidoethyldimethylamine, behenamidopropyldimethylamine, behenamidopropyldiethylamine, behenamidoethyldiethylamine, behenamidoethyldimethylamine, arachidamidopropyldimethylamine, arachidamidopropyldiethylamine, arachidamidoethyldiethylamine, arachidamidoethyldimethylamine, diethylaminoethylstearamide.

Useful amines in the present invention are disclosed in U.S. Pat. No. 4,275,055, Nachtigal, et al. These amines can also be used in combination with acids such as l-glutamic acid, lactic acid, hydrochloric acid, malic acid, succinic acid, acetic acid, fumaric acid, tartaric acid, citric acid, l-glutamic hydrochloride, maleic acid, and mixtures thereof; more preferably l-glutamic acid, lactic acid, citric acid. The amines herein are preferably partially neutralized with any of the acids at a molar ratio of the amine to the acid of from about 1:0.3 to about 1:2, more preferably from about 1:0.4 to about 1:1.

Di-Long Alkyl Quaternized Ammonium Salt

Di-long alkyl quaternized ammonium salt is preferably combined with a mono-long alkyl quaternized ammonium salt or mono-long alkyl amidoamine salt. It is believed that such combination can provide easy-to rinse feel, compared to single use of a monoalkyl quaternized ammonium salt or mono-long alkyl amidoamine salt. In such combination with a mono-long alkyl quaternized ammonium salt or mono-long alkyl amidoamine salt, the di-long alkyl quaternized ammonium salts are used at a level such that the wt % of the dialkyl quaternized ammonium salt in the cationic surfactant system is in the range of preferably from about 10% to about 50%, more preferably from about 30% to about 45%.

The dialkyl quaternized ammonium salt cationic surfactants useful herein are those having two long alkyl chains having 12-30 carbon atoms, preferably 16-24 carbon atoms, more preferably 18-22 carbon atoms. The remaining groups attached to nitrogen are independently selected from an alkyl group of from 1 to about 4 carbon atoms or an alkoxy, polyoxyalkylene, alkylamido, hydroxyalkyl, aryl or alkylaryl group having up to about 4 carbon atoms.

Di-long alkyl quaternized ammonium salts useful herein are those having the formula (II):

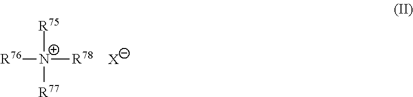

wherein two of R⁷⁵, R⁷⁶, R⁷⁷ and R⁷⁸ is selected from an alkyl group of from 12 to 30 carbon atoms or an aromatic, alkoxy, polyoxyalkylene, alkylamido, hydroxyalkyl, aryl or alkylaryl group having up to about 30 carbon atoms; the remainder of R⁷⁵, R⁷⁶, R⁷⁷ and R⁷⁸ are independently selected from an alkyl group of from 1 to about 4 carbon atoms or an alkoxy, polyoxyalkylene, alkylamido, hydroxyalkyl, aryl or alkylaryl group having up to about 4 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The alkyl groups can contain, in addition to carbon and hydrogen atoms, ether and/or ester linkages, and other groups such as amino groups. The longer chain alkyl groups, e.g., those of about 12 carbons, or higher, can be saturated or unsaturated. Preferably, one of R⁷⁵, R⁷⁶, R⁷⁷ and R⁷⁸ is selected from an alkyl group of from 12 to 30 carbon atoms, more preferably from 16 to 24 carbon atoms, still more preferably from 18 to 22 carbon atoms, even more preferably 22 carbon atoms; the remainder of R⁷⁵, R⁷⁶, R⁷⁷ and R⁷⁸ are independently selected from CH₃, C₂H₅, C₂H₄OH, and mixtures thereof; and X is selected from the group consisting of Cl, Br, CH₃OSO₃, C₂H₅OSO₃, and mixtures thereof.

Such dialkyl quaternized ammonium salt cationic surfactants include, for example, dialkyl (14-18) dimethyl ammonium chloride, ditallow alkyl dimethyl ammonium chloride, dihydrogenated tallow alkyl dimethyl ammonium chloride, distearyl dimethyl ammonium chloride, and dicetyl dimethyl ammonium chloride. Such dialkyl quaternized ammonium salt cationic surfactants also include, for example, asymmetric dialkyl quaternized ammonium salt cationic surfactants.

### C. High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound is included in the composition at a level of from about 0.1% to about 20%, preferably from about 1% to about 15%, more preferably from about 1.5% to about 8% by weight of the composition, in view of providing improved conditioning benefits such as slippery feel during the application to wet hair, softness and moisturized feel on dry hair.

### D. Aqueous Carrier

The gel matrix of the hair care composition of the present invention includes an aqueous carrier. Accordingly, the formulations of the present invention can be in the form of pourable liquids (under ambient conditions). Such compositions will therefore typically comprise an aqueous carrier, which is present at a level of from about 20 wt % to about 95 wt %, or even from about 60 wt % to about 85 wt %. The aqueous carrier may comprise water, or a miscible mixture of water and organic solvent, and in one aspect may comprise water with minimal or no significant concentrations of organic solvent, except as otherwise incidentally incorporated into the composition as minor ingredients of other components.

The aqueous carrier useful in the present invention includes water and water solutions of lower alkyl alcohols and polyhydric alcohols. The lower alkyl alcohols useful herein are monohydric alcohols having 1 to 6 carbons, in one aspect, ethanol and isopropanol. The polyhydric alcohols useful herein include propylene glycol, hexylene glycol, glycerin, and propane diol.

According to embodiments of the present invention, the hair care compositions may have a pH in the range from about 2 to about 10, at 25° C. In one embodiment, the hair care composition has a pH in the range from about 2 to about 6, which may help to solubilize minerals and redox metals already deposited on the hair. Thus, the hair care composition can also be effective toward washing out the existing minerals and redox metals deposits, which can reduce cuticle distortion and thereby reduce cuticle chipping and damage.

### E. Gel Matrix

The composition of the present invention comprises a gel matrix. The gel matrix comprises a cationic surfactant, a high melting point fatty compound, and an aqueous carrier. The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

### F. Additional Components

1. Silicone Conditioning Agent

According to embodiments of the present invention, the hair care composition includes a silicone conditioning agent which comprises a silicone compound. The silicone compound may comprise volatile silicone, non-volatile silicones, or combinations thereof. In one aspect, non-volatile silicones are employed. If volatile silicones are present, it will typically be incidental to their use as a solvent or carrier for commercially available forms of non-volatile silicone materials ingredients, such as silicone gums and resins. The silicone compounds may comprise a silicone fluid conditioning agent and may also comprise other ingredients, such as a silicone resin to improve silicone fluid deposition efficiency or enhance glossiness of the hair. The concentration of the silicone compound in the conditioner composition typically ranges from about 0.01 wt % to about 10 wt %, from about 0.1 wt % to about 8 wt %, from about 0.1 wt % to about 5 wt %, or even from about 0.2 wt % to about 3 wt %, for example

Exemplary silicone compounds include (a) a first polysiloxane which is non-volatile, substantially free of amino groups, and has a viscosity of from about 100,000 mm² s⁻¹ to about 30,000,000 mm² s⁻¹; (b) a second polysiloxane which is non-volatile, substantially free of amino groups, and has a viscosity of from about 5 mm² s⁻¹ to about 10,000 mm² s⁻¹; (c) an aminosilicone having less than about 0.5 wt % nitrogen by weight of the aminosilicone; (d) a silicone copolymer emulsion with an internal phase viscosity of greater than about 100×10⁶ mm² s⁻¹, as measured at 25° C.; (e) a silicone polymer containing quaternary groups; or (f) a grafted silicone polyol, wherein the silicone compounds (a)-(f) are disclosed in U.S. Patent Application Publication Nos. 2008/0292574, 2007/0041929, 2008/0292575, and 2007/0286837, each of which is incorporated by reference herein in its entirety.

a. First Polysiloxane

The hair care composition of the present invention may comprise a first polysiloxane. The first polysiloxane is non-volatile, and substantially free of amino groups. In the present invention, the first polysiloxanes being “substantially free of amino groups” means that the first polysiloxane contains 0 wt % of amino groups. The first polysiloxane has a viscosity of from about 100,000 mm² s⁻¹ to about 30,000,000 mm² s⁻¹ at 25° C. For example, the viscosity may range from about 300,000 mm² s⁻¹ to about 25,000,000 mm² s⁻¹, or from about 10,000,000 mm² s⁻¹ to about 20,000,000 mm² s⁻¹. The first polysiloxane has a molecular weight from about 100,000 to about 1,000,000. For example, the molecular weight may range from about 130,000 to about 800,000, or from about 230,000 to about 600,000. According to one aspect, the first polysiloxane may be nonionic.

Exemplary first non-volatile polysiloxanes useful herein include those in accordance with the following the general formula (I):

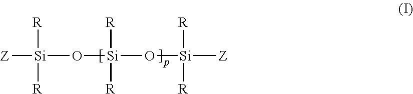

wherein R is alkyl or aryl, and p is an integer from about 1,300 to about 15,000, such as from about 1,700 to about 11,000, or from about 3,000 to about 8,000. Z represents groups which block the ends of the silicone chains. The alkyl or aryl groups substituted on the siloxane chain (R) or at the ends of the siloxane chains Z can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. According to an embodiment, suitable Z groups include hydroxy, methyl, methoxy, ethoxy, propoxy, and aryloxy. The two R groups on each silicon atom may represent the same group or different groups. According to one embodiment, the two R groups may represent the same group. Suitable R groups include methyl, ethyl, propyl, phenyl, methylphenyl and phenylmethyl. Exemplary silicone compounds include polydimethylsiloxane, polydiethylsiloxane, and polymethylphenylsiloxane. According to one embodiment, polydimethylsiloxane is the first polysiloxane. Commercially available silicone compounds useful herein include, for example, those available from the General Electric Company in their TSF451 series, and those available from Dow Corning in their Dow Corning SH200 series.

The silicone compounds that can be used herein also include a silicone gum. The term “silicone gum”, as used herein, means a polyorganosiloxane material having a viscosity at 25° C. of greater than or equal to 1,000,000 mm² s⁻¹. It is recognized that the silicone gums described herein can also have some overlap with the above-disclosed silicone compounds. This overlap is not intended as a limitation on any of these materials. The “silicone gums” will typically have a mass molecular weight in excess of about 165,000, generally between about 165,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. Commercially available silicone gums useful herein include, for example, TSE200A available from the General Electric Company.

b. Second Polysiloxane

The hair care composition of the present invention may comprise a second polysiloxane. The second polysiloxane is non-volatile, and substantially free of amino groups. In the present invention, the second polysiloxane being “substantially free of amino groups” means that the second polysiloxane contains 0 wt % of amino groups. The second polysiloxane has a viscosity of from about 5 mm² s⁻¹ to about 10,000 mm² s⁻¹ at 25° C., such as from about 5 mm² s⁻¹ to about 5,000 mm² s⁻¹, from about 10 mm² s⁻¹ to about 1,000 mm² s⁻¹, or from about 20 mm² s⁻¹ to about 350 mm² s⁻¹. The second polysiloxane has a molecular weight of from about 400 to about 65,000. For example, the molecular weight of the second polysiloxane may range from about 800 to about 50,000, from about 400 to about 30,000, or from about 400 to about 15,000. According to one aspect, the second polysiloxane may be nonionic. According to another aspect, the second polysiloxane may be a linear silicone.

Exemplary second non-volatile polysiloxanes useful herein include polyalkyl or polyaryl siloxanes in accordance with the following the general formula (II):

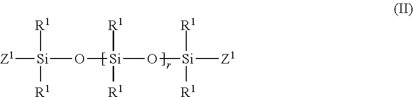

wherein R¹ is alkyl or aryl, and r is an integer from about 7 to about 850, such as from about 7 to about 665, from about 7 to about 400, or from about 7 to about 200. Z¹ represents groups which block the ends of the silicone chains. The alkyl or aryl groups substituted on the siloxane chain (R¹) or at the ends of the siloxane chains Z¹ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. According to an embodiment, suitable Z¹ groups include hydroxy, methyl, methoxy, ethoxy, propoxy, and aryloxy. The two R¹ groups on each silicon atom may represent the same group or different groups. According to one embodiment, the two R¹ groups may represent the same group. Suitable R¹ groups include methyl, ethyl, propyl, phenyl, methylphenyl and phenylmethyl. Exemplary silicone compounds include polydimethylsiloxane, polydiethylsiloxane, and polymethylphenylsiloxane. According to one embodiment, polydimethylsiloxane is the second polysiloxane. Commercially available silicone compounds useful herein include, for example, those available from the General Electric Company in their TSF451 series, and those available from Dow Corning in their Dow Corning SH200 series.

c. Aminosilicone

The hair care composition of the present invention may comprise an amino silicone having less than about 0.5 wt % nitrogen by weight of the aminosilicone, such as less than about 0.2 wt %, or less than about 0.1 wt %, in view of friction reduction benefit. It has been surprisingly found that higher levels of nitrogen (amine functional groups) in the amino silicone tend to result in less friction reduction, and consequently less conditioning benefit from the aminosilicone. The aminosilicone useful herein may have at least one silicone block with greater than 200 siloxane units, in view of friction reduction benefit. The aminosilicones useful herein include, for example, quaternized aminosilicone and non-quaternized aminosilicone.

In one embodiment, the aminosilicones useful herein are water-insoluble. In the present invention, “water-insoluble aminosilicone” means that the aminosilicone has a solubility of 10 g or less per 100 g water at 25° C., in another embodiment 5 g or less per 100 g water at 25° C., and in another embodiment 1 g or less per 100 g water at 25° C. In the present invention, “water-insoluble aminosilicone” means that the aminosilicone is substantially free of copolyol groups. If copolyol groups are present, they are present at a level of less than 10 wt %, less than 1 wt %, or less than 0.1 wt % by weight of the amionosilicone.

According to one embodiment, aminosilicone useful herein are those which conform to the general formula (III):

(R²)ₐG₃₋ₐ—Si(—O—SiG₂)ₙ(—O—SiG_{b}(R²)_{2-b})ₘ—O—SiG₃₋ₐ(R²)ₐ (IIII)

wherein G is hydrogen, phenyl, hydroxy, or C₁-C₈ alkyl, such as methyl; a is an integer having a value from 1 to 3, such as 1; b is an integer having a value from 0 to 2, such as 1; n is a number from 1 to 2,000, such as from 100 to 1,800, from 300 to 800, or from 500 to 600; m is an integer having a value from 0 to 1,999, such as from 0 to 10, or 0; R² is a monovalent radical conforming to the general formula C_{q}H_{2q}L, wherein q is an integer having a value from 2 to 8 and L is selected from the following groups: —N(R³₂)CH₂—CH₂—N(R³₂)₂; —N(R³)₂; —N⁺(R³)₃A⁻; —N(R³)CH₂—CH₂—N⁺R³H₂A⁻; wherein R³ is hydrogen, phenyl, benzyl, or a saturated hydrocarbon radical, such as an alkyl radical from about C₁ to about C₂₀; A⁻ is a halide ion. According to an embodiment, L is —N(CH₃)₂ or —NH₂. According to another embodiment, L is —NH₂.

The aminosilicone of the above formula is used at levels by weight of the composition of from about 0.1 wt % to about 5 wt %, alternatively from about 0.2 wt % to about 2 wt %, alternatively from about 0.2 wt % to about 1.0 wt %, and alternatively from about 0.3 wt % to about 0.8 wt %.

According to one embodiment, the aminosilicone may include those compounds corresponding to formula (III) wherein m=0; a=1; q=3; G=methyl; n is from about 1400 to about 1700, such as about 1600; and L is —N(CH₃)₂ or —NH₂, such as —NH₂. According to another embodiment, the aminosilicone may include those compounds corresponding to formula (III) wherein m=0; a=1; q=3; G=methyl; n is from about 400 to about 800, such as from about 500 to around 600; and L is L is —N(CH₃)₂ or —NH₂, such as —NH₂. Accordingly, the aforementioned aminosilicones can be called terminal aminosilicones, as one or both ends of the silicone chain are terminated by nitrogen containing group. Such terminal aminosilicones may provide improved friction reduction compared to graft aminosilicones.

Another example of an aminosilicone useful herein includes, for example, quaternized aminosilicone having a tradename KF8020 available from Shinetsu.

The above aminosilicones, when incorporated into the hair care composition, can be mixed with solvent having a lower viscosity. Such solvents include, for example, polar or non-polar, volatile or non-volatile oils. Such oils include, for example, silicone oils, hydrocarbons, and esters. Among such a variety of solvents, exemplary solvents include those selected from the group consisting of non-polar, volatile hydrocarbons, volatile cyclic silicones, non-volatile linear silicones, and mixtures thereof. The non-volatile linear silicones useful herein are those having a viscosity of from about 1 mm² s⁻¹ to about 20,000 mm² s⁻¹, such as from about 20 mm² s⁻¹ to about 10,000 mm² s⁻¹, at 25° C. According to one embodiment, the solvents are non-polar, volatile hydrocarbons, especially non-polar, volatile isoparaffins, in view of reducing the viscosity of the aminosilicones and providing improved hair conditioning benefits such as reduced friction on dry hair. Such mixtures may have a viscosity of from about 1,000 mPas to about 100,000 mPas, and alternatively from about 5,000 mPas to about 50,000 mPas.

d. Silicone Copolymer Emulsion

The hair care composition of the present invention may comprise a silicone copolymer emulsion with an internal phase viscosity of greater than about 100×10⁶ mm² s⁻¹. The silicone copolymer emulsion may be present in an amount of from about 0.1 wt % to about 15 wt %, alternatively from about 0.3 wt % to about 10 wt %, and alternatively about 0.5 wt % to about 5 wt %, by weight of the composition, in view of providing clean feel.

According to one embodiment, the silicone copolymer emulsion has a viscosity at 25° C. of greater than about 100×10⁶ mm² s⁻¹, alternatively greater than about 120×10⁶ mm² s⁻¹, and alternatively greater than about 150×10⁶ mm² s⁻¹. According to another embodiment, the silicone copolymer emulsion has a viscosity at 25° C. of less than about 1000×10⁶ mm² s⁻¹, alternatively less than about 500×10⁶ mm² s⁻¹, and alternatively less than about 300×10⁶ mm² s⁻¹. To measure the internal phase viscosity of the silicone copolymer emulsion, one may first break the polymer from the emulsion. By way of example, the following procedure can be used to break the polymer from the emulsion: 1) add 10 grams of an emulsion sample to 15 milliliters of isopropyl alcohol; 2) mix well with a spatula; 3) decant the isopropyl alcohol; 4) add 10 milliliters of acetone and knead polymer with spatula; 5) decant the acetone; 6) place polymer in an aluminum container and flatten/dry with a paper towel; and 7) dry for two hours in an 80° C. The polymer can then be tested using any known rheometer, such as, for example, a CarriMed, Haake, or Monsanto rheometer, which operates in the dynamic shear mode. The internal phase viscosity values can be obtained by recording the dynamic viscosity (n′) at a 9.900*10⁻³ Hz frequency point. According to one embodiment, the average particle size of the emulsions is less than about 1 micron, such as less than about 0.7 micron.

The silicone copolymer emulsions of the present invention may comprise a silicone copolymer, at least one surfactant, and water.

The silicone copolymer results from the addition reaction of the following two materials in the presence of a metal containing catalyst:

(i) a polysiloxane with reactive groups on both termini, represented by a general formula (IV):

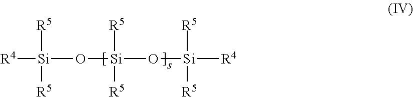

wherein:

R⁴ is a group capable of reacting by chain addition reaction such as, for example, a hydrogen atom, an aliphatic group with ethylenic unsaturation (i.e., vinyl, allyl, or hexenyl), a hydroxyl group, an alkoxyl group (i.e., methoxy, ethoxy, or propoxy), an acetoxyl group, or an amino or alkylamino group;

R⁵ is alkyl, cycloalkyl, aryl, or alkylaryl and may include additional functional groups such as ethers, hydroxyls, amines, carboxyls, thiols esters, and sulfonates; in an embodiment, R⁵ is methyl. Optionally, a small mole percentage of the groups may be reactive groups as described above for R⁵, to produce a polymer which is substantially linear but with a small amount of branching. In this case, the level of R⁵ groups equivalent to R⁴ groups may be less than about 10% on a mole percentage basis, such as less than about 2%;

s is an integer having a value such that the polysiloxane of formula (IV) has a viscosity of from about 1 mm² s⁻¹ to about 1×10⁶ mm² s⁻¹; and

(ii) at least one silicone compound or non-silicone compound comprising at least one or at most two groups capable of reacting with the R⁴ groups of the polysiloxane in formula (IV). According to one embodiment, the reactive group is an aliphatic group with ethylenic unsaturation.

The metal containing catalysts used in the above described reactions are often specific to the particular reaction. Such catalysts are known in the art. Generally, they are materials containing metals such as platinum, rhodium, tin, titanium, copper, lead, etc.

The mixture used to form the emulsion also may contain at least one surfactant. This can include non-ionic surfactants, cationic surfactants, anionic surfactants, alkylpolysaccharides, amphoteric surfactants, and the like. The above surfactants can be used individually or in combination.

An exemplary method of making the silicone copolymer emulsions described herein comprises the steps of 1) mixing materials (a) described above with material (b) described above, followed by mixing in an appropriate metal containing catalyst, such that material (b) is capable of reacting with material (a) in the presence of the metal containing catalyst; 2) further mixing in at least one surfactant and water; and 3) emulsifying the mixture. Methods of making such silicone copolymer emulsions are disclosed in U.S. Pat. No. 6,013,682; PCT Application No. WO 01/58986 A1; and European Patent Application No. EP0874017 A2.

A commercially available example of a silicone copolymer emulsion is an emulsion of about 60-70 wt % of divinyldimethicone/dimethicone copolymer having an internal phase viscosity of minimum 120×10⁶ mm² s⁻¹, available from Dow Corning with a tradename HMW2220.

e. Silicone Polymer Containing Quaternary Groups

The hair care composition of the present invention may comprise a silicone polymer containing quaternary groups (i.e., a quaternized silicone polymer). The quaternized silicone polymer provides improved conditioning benefits such as smooth feel, reduced friction, prevention of hair damage. Especially, the quaternary group can have good affinity with damaged/colorant hairs. The quaternized silicone polymer is present in an amount of from about 0.1 wt % to about 15 wt %, based on the total weight of the hair conditioning composition. For example, according to an embodiment, the quaternized silicone polymer may be present in an amount from about 0.2 wt % to about 10 wt %, alternatively from about 0.3 wt % to about 5 wt %, and alternatively from about 0.5 wt % to about 4 wt %, by weight of the composition.

The quaternized silicone polymer of the present invention is comprised of at least one silicone block and at least one non-silicone block containing quaternary nitrogen groups, wherein the number of the non-silicone blocks is one greater than the number of the silicone blocks. The silicone polymers correspond to the general structure (V):

A¹-B-(A²-B)ₘ-A¹ (V)

wherein, B is a silicone block having greater than 200 siloxane units; A¹ is an end group which may contain quaternary groups; A² is a non-silicone blocks containing quaternary nitrogen groups; and m is an integer 0 or greater, with the proviso that if m=0 then the A¹ group contains quaternary groups.

Structures corresponding to the general formula, for example, are disclosed in U.S. Pat. No. 4,833,225, in U.S. Patent Application Publication No. 2004/0138400, in U.S. Patent Application Publication No. 2004/0048996, and in U.S. Patent Application Publication No. 2008/0292575.

In one embodiment, the silicone polymers can be represented by the following structure (VI)

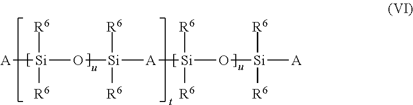

wherein, A is a group which contains at least one quaternary nitrogen group, and which is linked to the silicon atoms of the silicone block by a silicon-carbon bond, each A independently can be the same or different; R⁶ is an alkyl group of from about 1 to about 22 carbon atoms or an aryl group; each R⁶ independently can be the same or different; t is an integer having a value of from 0 or greater, for example t can be less than 20, or less than 10; and u is an integer greater than about 200, such as greater than about 250, or greater than about 300, and u may be less than about 700, or less than about 500. According to an embodiment, R⁶ is methyl.

f. Grafted Silicone Copolyol

The hair care composition of the present invention may comprise a grafted silicone copolyol in combination with the quaternized silicone polymer. It is believed that this grafted silicone copolyol can improve the spreadability of the quaternized silicone polymer by reducing the viscosity of the quaternized silicone polymer, and also can stabilize the quaternized silicone polymer in aqueous conditioner matrix. It is also believed that, by such improved spreadability, the hair care compositions of the present invention can provide better dry conditioning benefits such as friction reduction and/or prevention of damage with reduced tacky feel. It has been surprisingly found that the combination of the quaternized silicone polymer, grafted silicone copolyol, and cationic surfactant system comprising di-alkyl quaternized ammonium salt cationic surfactants provides improved friction reduction benefit, compared to a similar combination. Such similar combinations are, for example, a combination in which the grafted silicone copolyol is replaced with end-capped silicone copolyol, and another combination in which the cationic surfactant system is substantially free of di-alkyl quaternized ammonium salt cationic surfactants.

The grafted silicone copolyol is contained in the composition at a level such that the weight % of the grafted silicone copolyol to its mixture with quaternized silicone copolymer is in the range of from about 1 wt % to about 50 wt %, alternatively from about 5 wt % to about 40 wt %, and alternatively from about 10 wt % to 30 wt %.

The grafted silicone copolyols useful herein are those having a silicone backbone such as dimethicone backbone and polyoxyalkylene substitutions such as polyethylene oxide or/and polypropylene oxide substitutions. The grafted silicone copolyols useful herein have a hydrophilic-lipophilic balance (HLB) value of from about 5 to about 17, such as from about 8 to about 17, or from about 8 to about 12. The grafted silicone copolyols having the same INCI name have a variety of the weight ratio, depending on the molecular weight of the silicone portion and the number of the polyethylene oxide or/and polypropylene oxide substitutions.

According to an embodiment, exemplary commercially available grafted dimethicone copolyols include, for example: those having a tradename Silsoft 430 having an HLB value of from about 9 to about 12 (INCI name “PEG/PPG-20/23 dimethicone”) available from GE; those having a tradename Silsoft 475 having an HLB value of from about 13 to about 17 (INCI name “PEG-23/PPG-6 dimethicone”); those having a tradename Silsoft 880 having an HLB value of from about 13 to about 17 (INCI name “PEG-12 dimethicone”); those having a tradename Silsoft 440 having an HLB value of from about 9 to about 12 (INCI name “PEG-20/PPG-23 dimethicone”); those having a tradename DC5330 (INCI name “PEG-15/PPG-15 dimethicone”) available from Dow Corning.

The above quaternized silicone polymer and the grafted silicone copolyol may be mixed and emulsified by a emulsifying surfactant, prior to incorporating them into a gel matrix formed by cationic surfactants and high melting point fatty compounds, as discussed below. It is believed that, this pre-mixture can improve behavior of the quaternized silicone polymer and the grafted silicone copolyol, for example, increase the stability and reduce the viscosity to form more homogenized formulation together with the other components. Such emulsifying surfactant can be used at a level of about 0.001 wt % to about 1.5 wt %, alternatively from about 0.005% to about 1.0%, and alternatively from about 0.01 wt % to about 0.5 wt %, based on the total weight of the hair conditioning composition. Such surfactants may be nonionic, and have an HLB value of from about 2 to about 15, such as from about 3 to about 14, or from about 3 to about 10. Commercially available examples of emulsifying surfactant include nonionic surfactants having an INCI name C12-C14 Pareth-3 and having an HLB value of about 8 supplied from NIKKO Chemicals Co., Ltd. with tradename NIKKOL BT-3.

According to one embodiment, the hair care composition comprises a combination of two or more silicone conditioning agents, along with an EDDS sequestering agent and a gel matrix.

In one embodiment, the hair care composition comprises a polyalkylsiloxane mixture comprising (i) a first polyalkylsiloxane which is non-volatile, substantially free of amino groups, and has a viscosity of from about 100,000 mm² s⁻¹ to about 30,000,000 mm² s⁻¹, and (ii) a second polyalkylsiloxane which is non-volatile, substantially free of amino groups, and has a viscosity of from about 5 mm² s⁻¹ to about 10,000 mm² s⁻¹; an aminosilicone having less than about 0.5 wt % nitrogen by weight of the aminosilicone; and a silicone copolymer emulsion with an internal phase viscosity of greater than about 100×10⁶ mm² s⁻¹, as measured at 25° C. For example, in another embodiment, the hair care composition comprises from about 0.5 wt % to about 10 wt % of a polyalkylsiloxane mixture comprising (i) a first polyalkylsiloxane which is non-volatile, substantially free of amino groups, and has a viscosity of from about 100,000 mm² s⁻¹ to about 30,000,000 mm² s⁻¹, and (ii) a second polyalkylsiloxane which is non-volatile, substantially free of amino groups, and has a viscosity of from about 5 mm² s⁻¹ to about 10,000 mm² s⁻¹; from about 0.1 wt % to about 5 wt % of an aminosilicone having less than about 0.5 wt % nitrogen by weight of the aminosilicone; and from about 0.1 wt % to about 5 wt % of a silicone copolymer emulsion with an internal phase viscosity of greater than about 100×10⁶ mm² s⁻¹, as measured at 25° C.

In another embodiment, the hair care composition comprises a silicone polymer containing quaternary groups wherein said silicone polymer comprises silicone blocks with greater than about 200 siloxane units; and a grafted silicone copolyol. For example, in another embodiment, the hair care composition comprises from about 0.1 wt % to about 15 wt % of a silicone polymer containing quaternary groups wherein said silicone polymer comprises silicone blocks with greater than about 200 siloxane units; and a grafted silicone copolyol at a level such that the weight % of the grafted silicone copolyol in its mixture with the quaternized silicone polymer is in the range of from about 1 wt % to about 50 wt %.

In yet another embodiment, the hair care composition comprises an aminosilicone having a viscosity of from about 1,000 centistokes to about 1,000,000 centistokes, and less than about 0.5% nitrogen by weight of the aminosilicone; and (2) a silicone copolymer emulsion with an internal phase viscosity of greater than about 120×10⁶ centistokes, as measured at 25° C.

2. Other Conditioning Agents

Also suitable for use in the hair care compositions herein are the conditioning agents described by the Procter & Gamble Company in U.S. Pat. Nos. 5,674,478, and 5,750,122. Also suitable for use herein are those conditioning agents described in U.S. Pat. Nos. 4,529,586, 4,507,280, 4,663,158, 4,197,865, 4,217, 914, 4,381,919, and 4,422, 853.

a. Organic Conditioning Oils

The hair care compositions of the present invention may also further comprise an organic conditioning oil. According to embodiments of the present invention, the hair care composition may comprise from about 0.05 wt % to about 3 wt %, from about 0.08 wt % to about 1.5 wt %, or even from about 0.1 wt % to about 1 wt %, of at least one organic conditioning oil as the conditioning agent, in combination with other conditioning agents, such as the silicones (described herein). Suitable conditioning oils include hydrocarbon oils, polyolefins, and fatty esters. Suitable hydrocarbon oils include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils are typically from about C12 to about C19. Branched chain hydrocarbon oils, including hydrocarbon polymers, typically will contain more than 19 carbon atoms. Suitable polyolefins include liquid polyolefins, liquid poly-α-olefins, or even hydrogenated liquid poly-α-olefins. Polyolefins for use herein may be prepared by polymerization of C4 to about C14 or even C6 to about C12. Suitable fatty esters include, but are not limited to, fatty esters having at least 10 carbon atoms. These fatty esters include esters with hydrocarbyl chains derived from fatty acids or alcohols (e.g. mono-esters, polyhydric alcohol esters, and di- and tri-carboxylic acid esters). The hydrocarbyl radicals of the fatty esters hereof may include or have covalently bonded thereto other compatible functionalities, such as amides and alkoxy moieties (e.g., ethoxy or ether linkages, etc.).

3. Nonionic Polymers

The hair care composition of the present invention may also further comprise a nonionic polymer. According to an embodiment, the conditioning agent for use in the hair care composition of the present invention may include a polyalkylene glycol polymer. For example, polyalkylene glycols having a molecular weight of more than about 1000 are useful herein. Useful are those having the following general formula (VIII):

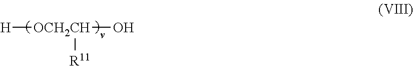

wherein R¹¹ is selected from the group consisting of H, methyl, and mixtures thereof; and v is the number of ethoxy units. The polyalkylene glycols, such as polyethylene glycols, can be included in the hair care compositions of the present invention at a level of from about 0.001 wt % to about 10 wt %. In an embodiment, the polyethylene glycol is present in an amount up to about 5 wt % based on the weight of the composition. Polyethylene glycol polymers useful herein are PEG-2M (also known as Polyox WSR® N-10, which is available from Union Carbide and as PEG-2,000); PEG-5M (also known as Polyox WSR® N-35 and Polyox WSR® N-80, available from Union Carbide and as PEG-5,000 and Polyethylene Glycol 300,000); PEG-7M (also known as Polyox WSR® N-750 available from Union Carbide); PEG-9M (also known as Polyox WSR® N-3333 available from Union Carbide); and PEG-14 M (also known as Polyox WSR® N-3000 available from Union Carbide).

4. Suspending Agent

The hair care compositions of the present invention may further comprise a suspending agent at concentrations effective for suspending water-insoluble material in dispersed form in the compositions or for modifying the viscosity of the composition. Such concentrations range from about 0.1 wt % to about 10 wt %, or even from about 0.3 wt % to about 5.0 wt %.

Suspending agents useful herein include anionic polymers and nonionic polymers. Useful herein are vinyl polymers such as cross linked acrylic acid polymers with the CTFA name Carbomer, cellulose derivatives and modified cellulose polymers such as methyl cellulose, ethyl cellulose, hydroxyethyl cellulose, hydroxypropyl methyl cellulose, nitro cellulose, sodium cellulose sulfate, sodium carboxymethyl cellulose, crystalline cellulose, cellulose powder, polyvinylpyrrolidone, polyvinyl alcohol, guar gum, hydroxypropyl guar gum, xanthan gum, arabia gum, tragacanth, galactan, carob gum, guar gum, karaya gum, carrageenan, pectin, agar, quince seed (Cydonia oblonga Mill), starch (rice, corn, potato, wheat), algae colloids (algae extract), microbiological polymers such as dextran, succinoglucan, pulleran, starch-based polymers such as carboxymethyl starch, methylhydroxypropyl starch, alginic acid-based polymers such as sodium alginate, alginic acid propylene glycol esters, acrylate polymers such as sodium polyacrylate, polyethylacrylate, polyacrylamide, polyethyleneimine, and inorganic water soluble material such as bentonite, aluminum magnesium silicate, laponite, hectonite, and anhydrous silicic acid.

Commercially available viscosity modifiers highly useful herein include Carbomers with trade names Carbopol® 934, Carbopol® 940, Carbopol® 950, Carbopol® 980, and Carbopol® 981, all available from B. F. Goodrich Company, acrylates/steareth-20 methacrylate copolymer with trade name ACRYSOL™ 22 available from Rohm and Hass, nonoxynyl hydroxyethylcellulose with trade name Amercell™ POLYMER HM-1500 available from Amerchol, methylcellulose with trade name BENECEL®, hydroxyethyl cellulose with trade name NATROSOL®, hydroxypropyl cellulose with trade name KLUCEL®, cetyl hydroxyethyl cellulose with trade name POLYSURF® 67, all supplied by Hercules, ethylene oxide and/or propylene oxide based polymers with trade names CARBOWAX® PEGs, POLYOX WASRs, and UCON® FLUIDS, all supplied by Amerchol.

Other optional suspending agents include crystalline suspending agents which can be categorized as acyl derivatives, long chain amine oxides, and mixtures thereof. These suspending agents are described in U.S. Pat. No. 4,741,855.

These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents.

Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA).

Examples of suitable long chain amine oxides for use as suspending agents include alkyl dimethyl amine oxides, e.g., stearyl dimethyl amine oxide.

Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

5. Deposition Aids

The hair care compositions of the present invention may further comprise a deposition aid, such as a cationic polymer. Cationic polymers useful herein are those having an average molecular weight of at least about 5,000, alternatively from about 10,000 to about 10 million, and alternatively from about 100,000 to about 2 million.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

The cationic polymer can be included in the hair care compositions of the present invention at a level of from about 0.001 wt % to about 10 wt %. In one embodiment, the cationic polymer is present in an amount up to about 5 wt % based on the weight of the composition.

### Deposition Polymer

In a further embodiment of the present invention, the composition of the present invention may further comprise a deposition polymer, preferable anionic/acid-deposition polymer. The deposition polymer is included at a level by weight of the composition of, from about 0.03% to about 8%, preferably from about 0.05% to about 3%, more preferably from about 0.1% to about 1%.

It is preferred that the weight ratio of (i) the deposition polymer to (ii) a sum of the mono-alkyl amine salt cationic surfactant, di-alkyl quaternized ammonium salt cationic surfactant, and high melting point fatty compound is from about 1:1 to about 1:160, more preferably from about 1:2.5 to about 1:120, still more preferably from about 1:3.5 to about 1:80. If the weight ratio of (i) to (ii) is too low, the composition may provide lower deposition of cationic surfactants, high melting point fatty compounds, and/or silicone compounds. If the weight ratio of (i) to (ii) is too high, the composition may influence rheology, and may undesirably decrease rheology of the composition.

The deposition polymer useful herein is a copolymer comprising: a vinyl monomer (A) with a carboxyl group in the structure; and a vinyl monomer (B) expressed by the following formula (1):

CH₂═C(R¹)—CO—X-(Q-O)ᵣ—R² (1)

wherein: R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O))ᵣ—R², the number of atoms bonded in a straight chain is 70 or less; and
wherein the vinyl monomer (A) is contained at a level of from about 10 mass % to about 50 mass %, and the vinyl monomer (B) is contained at level of from about 50 mass % to about 90 mass %.

### Vinyl Monomer (A)

The copolymer of the present invention contains a vinyl monomer (A) having a carboxyl group in the structure. The copolymer may contain one type of the vinyl monomer (A), or may contain two or more types of the vinyl monomer (A). The vinyl monomer (A) is preferably anionic.

This vinyl monomer (A) is contained at a level of from about 10 mass % based on the total mass of the copolymer, preferably from about 15 mass %, more preferably 20 mass % or higher, and even more preferably 25 mass % or higher, in view of improved deposition of cationic surfactants, fatty compounds and/or silicones, and to about 50 mass %, preferably 45 mass % or less, and more preferably 40 mass % or less, in view of not-deteriorating smoothness during application and/or the product viscosity.

Non-limited example of the vinyl monomer (A) having a carboxyl group include, for example, unsaturated carboxylic acid monomers having 3 to 22 carbon atoms. The unsaturated carboxylic acid monomer has, preferably 4 or more carbon atoms, and preferably 20 or less carbon atoms, more preferably 18 or less carbon atoms, still more preferably 10 or less carbon atoms, and even more preferably 6 or less carbon atoms. Furthermore, the number of carboxyl groups in the vinyl monomer (A) is preferably from 1 to 4, more preferably from 1 to 3, even more preferably from 1 to 2, and most preferably 1.

In view of improved deposition of cationic surfactants, fatty compounds and/or silicones, the vinyl monomer (A) is preferably an unsaturated carboxylic acid monomer expressed by the following formula (2) or formula (3), more preferably those expressed by the formula (2).

CH₂═C(R³)—CO—(O—(CH₂)ₘ—CO)ₙ—OH (2)

wherein: R³ represents a hydrogen atom or a methyl group, preferably a hydrogen atom; m represents an integer of 1 through 4, preferably 2 to 3; and n represents an integer of 0 through 4, preferably 0 to 2, and most preferably 0.

CH₂═C(R⁴)—COO—(CH₂)ₚ—OOC—(CH₂)_{q}—COOH (3)

wherein: R⁴ represents a hydrogen atom or a methyl group, preferably a hydrogen atom; p and q independently represent an integer of 2 through 6, preferably 2 to 3.

Examples of those expressed by the formula (2) include (meth)acrylic acid, crotonic acid, maleic acid, fumaric acid, itaconic acid, angelic acid, tiglic acid, 2-carboxy ethyl acrylate oligomer, and the like. Among them, preferred are acrylic acid and methacrylic acid, and more preferred is acrylic acid. Examples of those expressed by the formula (3) include acryloyloxy ethyl succinate, 2-methacryloyloxy ethyl succinate, and the like.

### Vinyl Monomer (B)

The copolymer contains a vinyl monomer (B). The copolymer may contain one type of the vinyl monomer (B), or may contain two or more types of the vinyl monomer (B). The vinyl monomer (B) is preferably nonionic.

The vinyl monomer (B) is contained at a level of from about 50 mass % based on the total mass of the copolymer in view of improving the feel and the smoothness during application, and to about 90 mass % based on the total mass of the copolymer, preferably to about 85 mass %, more preferably to about 80 mass %, still more preferably 75 mass %, in view of improved deposition of cationic surfactants, fatty compounds and/or silicones.

The Vinyl monomers (B) useful herein are those expressed by formula (4).

CH₂═C(R¹)—CO—X-(Q-O)ᵣ—R² (4)

wherein: R¹ represents a hydrogen atom or a methyl group; R² represents a hydrogen atom or an alkyl group with 1 through 5 carbon atoms, which may have a substitution group; Q represents an alkylene group with 2 through 4 carbon atoms which may also have a substitution group; r represents an integer from 2 through 15; and X represents an oxygen atom or an NH group; and in the structure -(Q-O)ᵣ—R², the number of atoms bonded in a straight chain is 70 or less.

If R² has a substitution group, the substitution group is a substitution group that does not react with other parts of the copolymer. The vinyl monomer (B) is preferably hydrophilic, and therefore R² is preferably a hydrogen atom or an alkyl group with 1˜3 carbon atoms, and more preferably a hydrogen atom or an alkyl group with 1 or 2 carbon atoms.

X preferably represents an oxygen atom.

Q represents preferably an alkylene group with 2 through 3 carbon atoms which may also have a substitution group, and more preferably an alkylene group with 2 through 3 carbon atoms without any substitution group. If the alkylene group of Q has a substitution group, it is preferred that such substitution group does not react with other parts of the copolymer, more preferably such substitution group has a molecular weight of 50 or less, still more preferably such substitution group has a molecular weight that is smaller than the structural moiety of -(Q-O)ᵣ—. Examples of such substitution group include a hydroxyl group, methoxy group, ethoxy group, and the like.

r represents preferably 3 or higher, and preferably 12 or less, in view of improved deposition of cationic surfactants, fatty compounds and/or silicones, and/or in view of smoothness during application.

As described above, in the structure -(Q-O)ᵣ—R², the number of atoms that are bonded by the straight chain is 70 or less. For example, if Q represents an n-butylene group, r=15, and R² represents an n-pentyl group, the number of atoms that are bonded in the straight chain of the structure -(Q-O)ᵣ—R² is calculated as 80, which therefore is outside of the scope. The number of atoms bonded in the straight chain in the structure -(Q-O)ᵣ—R² is preferably 60 or less, more preferably 40 or less, even more preferably 28 or less, and particularly preferably 20 or less, in view of improved deposition of cationic surfactants, fatty compounds and/or silicones, and/or in view of smoothness during application.

Examples of the vinyl monomer (B) include, methoxy polyethylene glycol (meth)acrylate (where the number of repetitions of polyethylene glycol (r in formula (4)) is between 2˜15), polyethylene glycol (meth)acrylate (where the number of repetitions of polyethylene glycol (r in formula (4)) is between 2˜15), methoxy polyethylene glycol/polypropylene glycol (meth)acrylate (where the number of repetitions of polyethylene glycol/polypropylene glycol (r in formula (4)) is between 2˜15), polyethylene glycol/polypropylene glycol (meth)acrylate (where the number of repetitions of polyethylene glycol/polypropylene glycol (r in formula (4)) is between 2˜15), methoxy polyethylene glycol/polybutylene glycol (meth)acrylate (where the number of repetitions of polyethylene glycol/polybutylene glycol (r in formula (4)) is between 2˜15), polyethylene glycol/polybutylene glycol (meth)acrylate (where the number of repetitions of polyethylene glycol/polybutylene glycol (r in formula (4)) is between 2˜15), methoxy polyethylene glycol (meth)acrylamide (where the number of repetitions of polyethylene glycol (r in formula (4)) is between 2˜15), and polyethylene glycol (meth)acrylamide (where the number of repetitions of polyethylene glycol (r in formula (4)) is between 2˜15); preferably methoxy polyethylene glycol (meth)acrylate (where the number of repetitions of polyethylene glycol (r in formula (4)) is between 3˜12), polyethylene glycol (meth)acrylate (where the number of repetitions of polyethylene glycol (r in formula (4)) is between 3˜12), methoxy polyethylene glycol/polypropylene glycol (meth)acrylate (where the number of repetitions of polyethylene glycol/polypropylene glycol (r in formula (4)) is between 3˜12), polyethylene glycol/polypropylene glycol (meth)acrylate (where the number of repetitions of polyethylene glycol/polypropylene glycol (r in formula (4)) is between 3˜12), methoxy polyethylene glycol/polybutylene glycol (meth)acrylate (where the number of repetitions of polyethylene glycol/polybutylene glycol (r in formula (4)) is between 3˜12), polyethylene glycol/polybutylene glycol (meth)acrylate (where the number of repetitions of polyethylene glycol/polybutylene glycol (r in formula (4)) is between 3˜12); more preferably methoxy polyethylene glycol (meth)acrylate (where the number of repetitions of polyethylene glycol (r in formula (4)) is between 3˜12), and polyethylene glycol (meth)acrylate (where the number of repetitions of polyethylene glycol (r in formula (4)) is between 3˜12).

### Vinyl Monomer (C)

In addition to the vinyl monomers (A) and (B), the copolymer may further contain a vinyl monomer (C) having an alkyl group with 12˜22 carbon atoms, in view of providing conditioning effect such as smoothness during application. When included, the amount of the vinyl monomer (C) is preferably 40 mass % or less, more preferably 30 mass % or less, even more preferably 25 mass % or less, and still more preferably 20 mass % or less based on the total mass of the copolymer, in view of improved deposition of cationic surfactants, fatty compounds and/or silicones, and/or in view of smoothness during application.

Preferably, the vinyl monomer (C) is a (meth)acrylate monomer having an alkyl group with 12˜22 carbon atoms, in view of smoothness during application. Furthermore, vinyl monomers with branched alkyl groups are particularly preferred.

Examples of the (meth)acrylate monomer having an alkyl group with 12˜22 carbon atoms include myristyl (meth)acrylate, isostearyl (meth)acrylate, stearyl (meth)acrylate, behenyl (meth)acrylate, cetyl (meth)acrylate, lauryl (meth)acrylate, synthetic lauryl (meth)acrylate, (however “synthetic lauryl (meth)acrylate” refers to an alkyl (meth)acrylate having alkyl groups with 12 carbon atoms and alkyl groups with 13 carbon atoms), and the like. Of these, (meth)acrylate monomers having an alkyl group with 12˜20 carbon atoms are preferable, and (meth)acrylate monomers having an alkyl group with 16˜18 carbon atoms are more preferable.

The copolymer may contain one type of the vinyl monomer (C), or may contain two or more types of the vinyl monomer (C).

### Other Monomers

In addition to the aforementioned vinyl monomers (A), (B), and (C), the copolymer may also contain other vinyl monomers, to the extent not to deteriorate the effect of the copolymer. Examples of other vinyl monomers include nonionic monomers, amphoteric monomers, semi-polar monomers, cationic monomers, as well as monomers containing a polysiloxane group, preferably nonionic monomers with or without polysiloxane group These other monomers are different from any of the aforementioned vinyl monomers (A), (B), and (C).

Normally the amount of such other monomers, if included, is 40 mass % or less of the total mass of the copolymer, preferably 30 mass % or less, more preferably 20 mass % or less, and even more preferably 10 mass % or less.

In view of improved deposition of cationic surfactants, fatty compounds, and/or silicones, the amount of cationic functional groups in the copolymer is preferably low, and for example cationic functional groups preferably account for 10 mole % or less of all functional groups in the copolymer. More preferably, the copolymer is free of cationic functional groups.

Examples of nonionic monomers include esters of (meth)acrylic acid and alcohols with 1˜22 carbon atoms, amides of (meth)acrylic acid and alkyl amines with 1˜22 carbon atoms, monoesters of (meth)acrylic acid and ethylene glycol, 1,3-propylene glycol or the like, as well as esters where the hydroxyl group of the monoester has been etherified by methanol, ethanol or the like, (meth)acryloyl morpholine and the like.

Examples of amphoteric monomers include (meth)acryl esters having a betaine group, (meth)acrylamide having a betaine group and the like.

Examples of semipolar monomers include (meth)acrylate esters having an amine oxide group, (meth)acrylamides having an amine oxide group, and the like.

Examples of cationic monomers include (meth)acrylate esters having a quaternary ammonium group, (meth)acrylamides having a quaternary ammonium group and the like.

The monomer containing a polysiloxane group is a monomer having a polysiloxane structure and also having a structure that can bond by covalent bond to the copolymer. These component units have high affinity towards silicone oil that is normally used in conjunction in cosmetic material compositions, and are thought to act by bonding the silicone oil to the other component units in the copolymer and thus increasing the adsorption force of silicone oil to the skin and hair, particularly damaged hair.

The polysiloxane structure is a structure where two or more repeating structural units expressed by the following formula (4) are linked.

—(SiR⁵R⁶—O)— (4)

In formula (4), R⁵ and R⁶ independently represent an alkyl group with 1 to 3 carbon atoms or a phenyl group.

The structure that can link via covalent bond to the copolymer can be a structure that has a vinyl structure such as a (meth)acrylate ester, or (meth)acrylamide and that can copolymerize with another monomer, a structure that has a functional group such as a thiol, that can link to the copolymer by chain transfer during polymerization, or a structure that has an isocyanate group, carboxylic acid group, hydroxyl group, amino group, or the like, and that can react and link to the functional groups on the copolymer, but there is no restriction to these structures.

A plurality of these linkable structures can be present in one monomer containing a polysiloxane group. In the copolymer, the polysiloxane structure can link by a graft structure to the main chain, or conversely the polysiloxane structure can be the main chain with the other structure link by a graft structure, and in addition the polysiloxane structure and the other structure can be linked in a straight chain condition by a block structure.

The monomer containing a polysiloxane group is preferably expressed by the following formula (5).

CH₂═C(R⁷)—Z—(SiR⁸R⁹—O)ₛ—R¹⁰ (5)

In the formula, R⁷ represents a hydrogen atom or a methyl group, R⁸ and R⁹ independently represent an alkyl group with 1 to 3 carbon atoms or a phenyl group, R¹⁰ represents an alkyl group with 1 to 8 carbon atoms, Z represents a bivalent linking group or a direct bond, and s represents an integer between 2 to 200.

More preferably, s is 3 or higher, and even more preferably, s is 5 or higher, in view of increased affinity to silicone oil, and preferably s is 50 or less, in view of enhanced copolymerization with the other monomers.

Z represents a bivalent linking group or a direct bond, but a linking group containing one or a combination of two or more of the structures suggested below is preferable. The numbers that are combined is not particularly restricted, but normally is 5 or less. Furthermore, the direction of the following structures are arbitrary (the polysiloxane group side can be on either end). Note, in the following, R represents an alkylene group with 1 to 6 carbon atoms or a phenylene group:

—COO—R—; —CONH—R—; —O—R—; —R—

The monomer expressed by the aforementioned formula (5), include, for example, α-(vinyl phenyl) polydimethyl siloxane, α-(vinyl benzyloxy propyl) polydimethyl siloxane, α-(vinyl benzyl) polymethyl phenyl siloxane, α-(methacryloyl oxypropyl) polydimethyl siloxane, α-(methacryloyloxy propyl) polymethyl phenyl siloxane, α-(methacryloyl amino propyl) polydimethyl siloxane and the like. The monomer containing a polysiloxane group can be a single type, or can be two or more types used in combination.

In order to adjust the molecular weight and the viscosity of the copolymer, a cross-linking agent such as a polyfunctional acrylate or the like can be introduced to the copolymer. However, in this invention, it is preferred that a cross-linking agent is not included in the copolymer.

### Structure Analysis

The amount of the vinyl monomers (A), (B), and (C) as well as other monomers in the copolymer can be measured using IR absorption or Raman scattering by the carbonyl groups, amide bonds, polysiloxane structures, various types of functional groups, carbon backbone and the like, by ¹H-NMR of methyl groups in the polydimethyl siloxane, amide bond sites, and methyl groups and methylene groups adjacent thereto, as well as various types of NMR represented by ¹³C-NMR and the like.

### Weighted Average Molecular Weight

The weighted average molecular weight of the copolymer is preferably 3,000 or higher, more preferably 5,000 or higher, and even more preferably 10,000 or higher, in view of providing conditioning effect via foaming a complex with cationic surfactant, and preferably to about 2,000,000, more preferably 1,000,000 or less, still more preferably 500,000 or less, even more preferably 100,000 or less, and most preferably 50,000 or less, in view of feeling after drying.

The weighted average molecular weight of the copolymer can be measured by gel permeation chromatography (GPC). The development solvent that is used in gel permeation chromatography is not particularly restricted so long as being a normally used solvent, but for example, the measurement can be performed using a solvent blend of water/methanol/acetic acid/sodium acetate.

### Viscosity

The copolymer preferably has a viscosity for a 50 mass % of an aqueous carrier solution of lower alkyl alcohols and polyhydric alcohols, preferably ethanol aqueous solution, more preferably butanediol aqueous solution at 25° C. of 5 mPa·s or higher and 50,000 mPa·s or less. The viscosity is more preferably 10 mPa·s or higher, even more preferably 15 mPa·s or higher, but on the other hand is more preferably 10,000 mPa·s or less, and even more preferably 5,000 mPa·s or less. The viscosity of the copolymer is preferably 5 mPa·s or higher and 50,000 mPa·s or less, from the perspective of handling. The viscosity can be measured using a BL-type viscometer.

Similar to the weighted average molecular weight, the viscosity of the copolymer can be adjusted by controlling the degree of polymerization of the copolymer, and can be controlled by increasing or decreasing the amount of a cross-linking agent such as a polyfunctional acrylate or the like that is added.

6. Benefit Agents

In an embodiment, the hair care composition further comprises one or more additional benefit agents. The benefit agents comprise a material selected from the group consisting of anti-dandruff agents, vitamins, lipid soluble vitamins, chelants, perfumes, brighteners, enzymes, sensates, attractants, anti-bacterial agents, dyes, pigments, bleaches, and mixtures thereof.

In one aspect said benefit agent may comprise an anti-dandruff agent. Such anti-dandruff particulate should be physically and chemically compatible with the components of the composition, and should not otherwise unduly impair product stability, aesthetics or performance.

According to an embodiment, the hair care composition comprises an anti-dandruff active, which may be an anti-dandruff active particulate. In an embodiment, the anti-dandruff active is selected from the group consisting of: pyridinethione salts; azoles, such as ketoconazole, econazole, and elubiol; selenium sulphide; particulate sulfur; keratolytic agents such as salicylic acid; and mixtures thereof. In an embodiment, the anti-dandruff particulate is a pyridinethione salt.

Pyridinethione particulates are suitable particulate anti-dandruff actives. In an embodiment, the anti-dandruff active is a 1-hydroxy-2-pyridinethione salt and is in particulate form. In an embodiment, the concentration of pyridinethione anti-dandruff particulate ranges from about 0.01 wt % to about 5 wt %, or from about 0.1 wt % to about 3 wt %, or from about 0.1 wt % to about 2 wt %. In an embodiment, the pyridinethione salts are those formed from heavy metals such as zinc, tin, cadmium, magnesium, aluminium and zirconium, generally zinc, typically the zinc salt of 1-hydroxy-2-pyridinethione (known as “zinc pyridinethione” or “ZPT”), commonly 1-hydroxy-2-pyridinethione salts in platelet particle form. In an embodiment, the 1-hydroxy-2-pyridinethione salts in platelet particle form have an average particle size of up to about 20 microns, or up to about 5 microns, or up to about 2.5 microns. Salts formed from other cations, such as sodium, may also be suitable. Pyridinethione anti-dandruff actives are described, for example, in U.S. Pat. No. 2,809,971; U.S. Pat. No. 3,236,733; U.S. Pat. No. 3,753,196; U.S. Pat. No. 3,761,418; U.S. Pat. No. 4,345,080; U.S. Pat. No. 4,323,683; U.S. Pat. No. 4,379,753; and U.S. Pat. No. 4,470,982.

In an embodiment, in addition to the anti-dandruff active selected from polyvalent metal salts of pyrithione, the composition further comprises one or more anti-fungal and/or anti-microbial actives. In an embodiment, the anti-microbial active is selected from the group consisting of: coal tar, sulfur, fcharcoal, whitfield's ointment, castellani's paint, aluminum chloride, gentian violet, octopirox (piroctone olamine), ciclopirox olamine, undecylenic acid and its metal salts, potassium permanganate, selenium sulphide, sodium thiosulfate, propylene glycol, oil of bitter orange, urea preparations, griseofulvin, 8-hydroxyquinoline ciloquinol, thiobendazole, thiocarbamates, haloprogin, polyenes, hydroxypyridone, morpholine, benzylamine, allylamines (such as terbinafine), tea tree oil, clove leaf oil, coriander, palmarosa, berberine, thyme red, cinnamon oil, cinnamic aldehyde, citronellic acid, hinokitol, ichthyol pale, Sensiva SC-50, Elestab HP-100, azelaic acid, lyticase, iodopropynyl butylcarbamate (IPBC), isothiazalinones such as octyl isothiazalinone, and azoles, and mixtures thereof. In an embodiment, the anti-microbial is selected from the group consisting of: itraconazole, ketoconazole, selenium sulphide, coal tar, and mixtures thereof.

In an embodiment, the azole anti-microbials is an imidazole selected from the group consisting of: benzimidazole, benzothiazole, bifonazole, butaconazole nitrate, climbazole, clotrimazole, croconazole, eberconazole, econazole, elubiol, fenticonazole, fluconazole, flutimazole, isoconazole, ketoconazole, lanoconazole, metronidazole, miconazole, neticonazole, omoconazole, oxiconazole nitrate, sertaconazole, sulconazole nitrate, tioconazole, thiazole, and mixtures thereof, or the azole anti-microbials is a triazole selected from the group consisting of: terconazole, itraconazole, and mixtures thereof. When present in the hair care composition, the azole anti-microbial active is included in an amount of from about 0.01 wt % to about 5 wt %, or from about 0.1 wt % to about 3 wt %, or from about 0.3 wt % to about 2 wt %. In an embodiment, the azole anti-microbial active is ketoconazole. In an embodiment, the sole anti-microbial active is ketoconazole.

Embodiments of the hair care composition may also comprise a combination of anti-microbial actives. In an embodiment, the combination of anti-microbial active is selected from the group of combinations consisting of: octopirox and zinc pyrithione, pine tar and sulfur, salicylic acid and zinc pyrithione, salicylic acid and elubiol, zinc pyrithione and elubiol, zinc pyrithione and climbasole, octopirox and climbasole, salicylic acid and octopirox, and mixtures thereof.

In an embodiment, the composition comprises an effective amount of a zinc-containing layered material. In an embodiment, the composition comprises from about 0.001 wt % to about 10 wt %, or from about 0.01 wt % to about 7 wt %, or from about 0.1 wt % to about 5 wt % of a zinc-containing layered material, by total weight of the composition.

Zinc-containing layered materials may be those with crystal growth primarily occurring in two dimensions. It is conventional to describe layer structures as not only those in which all the atoms are incorporated in well-defined layers, but also those in which there are ions or molecules between the layers, called gallery ions (A. F. Wells “Structural Inorganic Chemistry” Clarendon Press, 1975). Zinc-containing layered materials (ZLMs) may have zinc incorporated in the layers and/or be components of the gallery ions. The following classes of ZLMs represent relatively common examples of the general category and are not intended to be limiting as to the broader scope of materials which fit this definition.

Many ZLMs occur naturally as minerals. In an embodiment, the ZLM is selected from the group consisting of: hydrozincite (zinc carbonate hydroxide), aurichalcite (zinc copper carbonate hydroxide), rosasite (copper zinc carbonate hydroxide), and mixtures thereof. Related minerals that are zinc-containing may also be included in the composition. Natural ZLMs can also occur wherein anionic layer species such as clay-type minerals (e.g., phyllosilicates) contain ion-exchanged zinc gallery ions. All of these natural materials can also be obtained synthetically or formed in situ in a composition or during a production process.

Another common class of ZLMs, which are often, but not always, synthetic, is layered double hydroxides. In an embodiment, the ZLM is a layered double hydroxide conforming to the formula [M²⁺₁₋ₓM³⁺ₓ(OH)₂]^{x+}A^{m-}_{x/m}.nH₂O wherein some or all of the divalent ions (M²⁺) are zinc ions (Crepaldi, E L, Pava, P C, Tronto, J, Valim, J B J. Colloid Interfac. Sci. 2002, 248, 429-42).

Yet another class of ZLMs can be prepared called hydroxy double salts (Morioka, H., Tagaya, H., Karasu, M, Kadokawa, J, Chiba, K Inorg. Chem. 1999, 38, 4211-6). In an embodiment, the ZLM is a hydroxy double salt conforming to the formula [M²⁺₁₋ₓM²⁺₁₊ₓ(OH)_{3(1-y)}]⁺Aⁿ⁻_{(1=3y)/n}.nH₂O where the two metal ions (M²⁺) may be the same or different. If they are the same and represented by zinc, the formula simplifies to [Zn₁₊ₓ(OH)₂]^{2x+}2xA⁻.nH₂O. This latter formula represents (where x=0.4) materials such as zinc hydroxychloride and zinc hydroxynitrate. In an embodiment, the ZLM is zinc hydroxychloride and/or zinc hydroxynitrate. These are related to hydrozincite as well wherein a divalent anion replace the monovalent anion. These materials can also be formed in situ in a composition or in or during a production process.

In embodiments having a zinc-containing layered material and a pyrithione or polyvalent metal salt of pyrithione, the ratio of zinc-containing layered material to pyrithione or a polyvalent metal salt of pyrithione is from about 5:100 to about 10:1, or from about 2:10 to about 5:1, or from about 1:2 to about 3:1.

The on-scalp deposition of the anti-dandruff active is at least about 1 microgram/cm². The on-scalp deposition of the anti-dandruff active is important in view of ensuring that the anti-dandruff active reaches the scalp where it is able to perform its function. In an embodiment, the deposition of the anti-dandruff active on the scalp is at least about 1.5 microgram/cm², or at least about 2.5 microgram/cm², or at least about 3 microgram/cm², or at least about 4 microgram/cm², or at least about 6 microgram/cm², or at least about 7 microgram/cm², or at least about 8 microgram/cm², or at least about 8 microgram/cm², or at least about 10 microgram/cm². The on-scalp deposition of the anti-dandruff active is measured by having the hair of individuals washed with a composition comprising an anti-dandruff active, for example a composition pursuant to the present invention, by trained a cosmetician according to a conventional washing protocol. The hair is then parted on an area of the scalp to allow an open-ended glass cylinder to be held on the surface while an aliquot of an extraction solution is added and agitated prior to recovery and analytical determination of anti-dandruff active content by conventional methodology, such as HPLC.

### Test Methods

### A. Molecular Weight Distribution

The weight average molecular weight (Mw) is measured using gel permeation chromatography (GPC) and multi-angle laser light scattering (MALLS). The GPC/MALLS system used for the analysis is comprised of a Waters Alliance e2695 Separations Module, a Waters 2414 interferometric refractometer, and a Wyatt Heleos II 18 angle laser light scattering detector. The column set used for separation is purchased from TOSOH Biosciences LLC, King of Prussia, Pa. and included: Guard Column TSKgel G1000Hx-GMHxl-L (Cat #07113), TSKgel G3000Hx1 (Cat #0016136), TSKgel G2500Hx1 (Cat #0016135), and TSKgel G2000Hx1 (Cat #0016134). Wyatt ASTRA 6 software was used for instrument operation and data analysis. The 90 degree light scattering detection angle is calibrated using filtered, anhydrous toluene. The remaining detection angles are normalized using an isotropic scatterer in THF. To verify instrument performance of the MALLS and RI (refractive index) detectors, a poly(styrene) standard with a known Mw and known dn/dc (in the mobile phase) is run. Acceptable performance of the MALLS and RI detectors gives a calculated Mw within 5% of the reported Mw of the poly(styrene) standard and a mass recovery between 95 and 105%.

To complete the GPC/MALLS analysis, a value of dn/dc is needed. The value of dn/dc is measured as follows. The RI detector is thermostated to 35° C. A series of five concentration standards of the metathesized unsaturated polyol ester in THF is prepared in the range 0.5 mg/ml to 5.5 mg/ml. A THF blank is injected directly into the refractive index detector, followed by each of the metathesized unsaturated polyol ester concentration standards, and ending with another THF blank. The volume of each sample injected is large enough to obtain a flat plateau region of constant differential refractive index versus time; a value of 1.0 ml is typically used. In the ASTRA software, a baseline is constructed from the initial and final THF injections. For each sample, peak limits are defined and the concentrations entered to calculate dn/dc in the ASTRA software. For the metathesized canola oil of Example 2 in THF, a dn/dc value of 0.072 ml/g is obtained.

For the GPC/MALLS analysis of a metathesized unsaturated polyol ester, a total of three samples are evaluated: the metathesized unsaturated polyol ester, a non-metathesized unsaturated polyol ester (glycerol trioleate [122-32-7], Sigma-Aldrich, Milwaukee, Wis.), and a representative olefin (1-octadecene, [112-88-9], Sigma-Aldrich, Milwaukee, Wis.). The GPC samples are dissolved in tetrahydrofuran (THF). Concentrations for the metathesized unsaturated polyol ester are approximately 20 mg/ml, and concentrations for the non-metathesized unsaturated polyol ester and olefin are approximately 5 mg/ml. After all the material is dissolved, each solution is filtered by a 0.45 micron nylon filter disk into a GPC autosampler vial for analysis. The GPC column temperature is at room temperature, approximately 25° C. HPLC grade THF is used as the mobile phase and is delivered at a constant flow rate of 1.0 ml/min. The injection volume is 100 microliters and the run time is 40 minutes. Baselines are constructed for all signals. Peak elution limits include metathesized unsaturated polyol ester and non-metathesized unsaturated polyol ester, but exclude later eluting residual olefin. The retention times of the non-metathesized unsaturated polyol ester and olefin were determined from the separate injection runs of both the non-metathesized unsaturated polyol ester and olefin. Baselines and scattering detectors are reviewed.

### B. Oligomer Index

The oligomer index of the metathesized unsaturated polyol ester is calculated from data that is determined by Supercritical Fluid Chromatography-Fourier Transform Orbital Trapping Mass Spectrometry (SFC-Orbitrap MS). The sample to be analyzed is typically dissolved in methylene chloride or a methylene chloride-hexane mixture at a concentration of 1000 ppm (1 mg/mL). A further 25×-100× dilution is typically made into hexane (for a final concentration of 10-40 ppm). A volume of 2-7.5 μL is typically injected on to a SFC column (for example, a commercially available 3 mm i.d.×150 mm Ethylpyridine column, 3 μM particle size).

During the chromatography run, the mobile phase is typically programmed from 100% carbon dioxide with a gradient of one percent per minute methanol. The effluent from the column is directed to a mixing tee where an ionization solution is added. The ionization medium is typically 20 mM ammonium formate in methanol at a flow of 0.7 mL/min while the SFC flow is typically 1.6 mL/min into the tee. The effluent from the mixing tee enters the ionization source of the Orbitrap Mass Spectrometer, which is operated in the heated electrospray ionization mode at 320° C.

In one aspect, a hybrid linear ion trap—Orbitrap mass spectrometer (i.e., the Orbitrap Elite from Thermoelectron Corp.) is calibrated and tuned according to the manufacturer's guidelines. A mass resolution (m/Δm peak width at half height) from 100,000 to 250,000 is typically used. C,H,O compositions of eluting species (typically associated with various cations, e.g., NH₄⁺, H⁺, Na⁺) are obtained by accurate mass measurement (0.1-2 ppm) and are correlated to metathesis products. Also, sub-structures may be probed by linear ion trap “MSⁿ” experiments with subsequent accurate-mass analysis in the Orbitrap, as practiced typically in the art.

The metathesis monomers, dimers, trimers, tetramers, pentamers, and higher order oligomers are fully separated by SFC. The chromatogram based on ion current from the Orbitrap MS may be integrated, as typically practiced in the art, for each of the particular oligomer groups including metathesis monomers, metathesis dimers, metathesis trimers, metathesis pentamers, and each of the higher order oligomers. These raw areas may then be formulated into various relative expressions, based on normalization to 100%. The sum of the areas of metathesis trimers through the highest oligomer detected is divided by the sum of all metathesis species detected (metathesis monomers to the highest oligomer detected). This ratio is called the “Oligomer Index”. As used herein, the Oligomer Index” is a relative measure of the fraction of the metathesized unsaturated polyol ester which is comprised of trimers, tetramers, pentamers, and higher order oligomers.

### C. Iodine Value

Another aspect of the invention provides a method to measure the iodine value of the metathesized unsaturated polyol ester. The iodine value is determined using AOCS Official Method Cd 1-25 with the following modifications: carbon tetrachloride solvent is replaced with chloroform (25 ml), an accuracy check sample (oleic acid 99%, Sigma-Aldrich; IV=89.86±2.00 cg/g) is added to the sample set, and the reported IV is corrected for minor contribution from olefins identified when determining the free hydrocarbon content of the metathesized unsaturated polyol ester.

### D. Free Hydrocarbon Content

Another aspect of this invention provides a method to determine the free hydrocarbon content of the metathesized unsaturated polyol ester. The method combines gas chromatography/mass spectroscopy (GC/MS) to confirm identity of the free hydrocarbon homologs and gas chromatography with flame ionization detection (GC/FID) to quantify the free hydrocarbon present.

Sample Prep: The sample to be analyzed was typically trans-esterified by diluting (e.g. 400:1) in methanolic KOH (e.g. 0.1N) and heating in a closed container until the reaction was complete (i.e. 90° C. for 30 min.) then cooled to room temperature. The sample solution could then be treated with 15% boron tri-fluoride in methanol and again heated in a closed vessel until the reaction was complete (i.e. at 60° C. for 30 min.) both to acidify (methyl orange-red) and to methylate any free acid present in the sample. After allowing to cool to room temperature, the reaction was quenched by addition of saturated NaCl in water. An organic extraction solvent such as cyclohexane containing a known level internal standard (e.g. 150 ppm dimethyl adipate) was then added to the vial and mixed well. After the layers separated, a portion of the organic phase was transferred to a vial suitable for injection to the gas chromatograph. This sample extraction solution was analyzed by GC/MS to confirm identification of peaks matching hydrocarbon retention times by comparing to reference spectra and then by GC/FID to calculate concentration of hydrocarbons by comparison to standard FID response factors.

A hydrocarbon standard of known concentrations, such as 50 ppm each, of typically observed hydrocarbon compounds (i.e. 1-dodecene, 1-tridecene, 1-tetradecene, 1-pentadecene, 1-hexadecene, 1-heptadecene, 1-octadecene, dodecane, tridecane, tetradecane, pentadecane, hexadecane, heptadecane and octadecane) was prepared by dilution in the same solvent containing internal standard as was used to extract the sample reaction mixture. This hydrocarbon standard was analyzed by GC/MS to generate retention times and reference spectra and then by GC/FID to generate retention times and response factors.

GC/MS:

An Agilent 7890 GC equipped with a split/splitless injection port coupled with a Waters QuattroMicroGC mass spectrometer set up in EI+ ionization mode was used to carry out qualitative identification of peaks observed. A non-polar DB1-HT column (15 m×0.25 mm×0.1 um de was installed with 1.4 mL/min helium carrier gas. In separate runs, 1 uL of the hydrocarbon standard and sample extract solution were injected to a 300° injection port with a split ratio of 25:1. The oven was held at 40° C. for 1 minute then ramped 15 C°/minute to a final temperature of 325° C. which was held for 10 minutes resulting in a total run time of 30 minutes. The transfer line was kept at 330° C. and the temperature of the EI source was 230° C. The ionization energy was set at 70 eV and the scan range was 35-550 m/z.

GC/FID:

An Agilent 7890 GC equipped with a split/splitless injection port and a flame ionization detector was used for quantitative analyses. A non-polar DB1-HT column (5 m×0.25 mm×0.1 um df) was installed with 1.4 mL/min helium carrier gas. In separate runs, 1 uL of the hydrocarbon standard and sample extract solution was injected to a 330° injection port with a split ratio of 100:1. The oven was held at 40° C. for 0.5 minutes then ramped at 40 C°/minute to a final temperature of 380° C. which was held for 3 minutes resulting in a total run time of 12 minutes. The FID was kept at 380° C. with 40 mL/minute hydrogen gas flow and 450 mL/min air flow. Make up gas was helium at 25 mL/min. The hydrocarbon standard was used to create a calibration table in the Chemstation Data Analysis software including known concentrations to generate response factors. These response factors were applied to the corresponding peaks in the sample chromatogram to calculate total amount of free hydrocarbon found in each sample.

### E. Wet and Dry Combing Test Method

This test method is designed to allow for a subjective evaluation of the basic performance of rinse-off conditioners for both wet combing and dry combing efficacy. In a typical test, 3 to 5 separate formulations may be assessed for their performance. The assessment may include control treatments containing no silicone and an elevated silicone level to facilitate differentiation of performance. The substrate is virgin brown hair obtainable from a variety of sources that is screened to insure uniformity and lack of meaningful surface damage or low lift bleach damaged hair.

a. Treatment Procedure

Four to five 4 gram, 8 inch length switches are combined in a hair switch holder, wet for ten seconds with manipulation with 39±1° C. water of medium hardness (3-10 gpg) to ensure complete and even wetting. The switch is deliquored lightly and Clarifying shampoo is applied uniformly over the length of the combined switches from one inch below the holder towards the tip at a level of 0.1 gram product per one gram of dry hair (0.1 g/g of hair or 2 g for 20 g hair). The switch combo is lathered for 30 seconds by a rubbing motion typical of that used by consumers and rinsed with 39±1° C. water flowing at 1.5 gal/min (with the hair being manipulated) for a further 30 seconds to ensure completeness. This step is repeated. The conditioner treatments are applied in the same way as shampoo above (0.1 g/g of hair or reduced to 0.05 g/g of hair for more concentrated prototypes), milked throughout the switch combo for 30 seconds, left to sit for a further 30 seconds, and rinsed thoroughly with manipulation, again for 30 seconds. The switches are deliquored lightly, separated from each other, hung on a rack so that they are not in contact, and detangled with a wide tooth comb.

b. Grading Procedures

For wet combing evaluations using trained graders, the switches are separated on the rack into the five sets with one switch from each treatment included in the grading set. Only two combing evaluations are performed on each switch. The graders are asked to compare the treatments by combing with a narrow tooth nylon comb typical of those used by consumers and rate the ease/difficulty on a zero to ten scale. Ten separate evaluations are collected and the results analyzed by a statistical analysis package for establishing statistical significance. Statistical significance in differences between treatments is determined using Statgraphics Plus 5.1.

For dry combing evaluations, the switches from above are moved into a controlled temperature and humidity room (22° C./50% RH) and allowed to dry overnight. They remain separated as above and panelists are requested to evaluate dry conditioning performance by making three assessments; dry combing ease of the middle of the switch, dry combing ease of the tips, and a tactile assessment of tip feel. The same ten point scale is used for these comparisons. Again, only two panelists make an assessment of each switch set. Statistical analysis to separate differences is performed using the same method as above.

### F. Friction Reduction on Dry Hair (IFM)

Dry conditioning performance is also evaluated via hair friction force measurements with an Instron Tester instrument (Instron 5542, Instron, Inc.; Canton, Mass., USA). In a typical procedure, hair switches are first prepared according to treatment protocol C and dried overnight in a controlled temperature and humidity room (22° C./50% RH). The friction force (grams) between the hair surface and a urethane pad along the hair is measured, with three measurements per switch.

### G. Wet Conditioning Tests

This rinse friction test determines the amount of conditioning provided by hair care composition products as measured by the force required to pull hair through an Instron while wet. The operator ranks and balances the 4 g, 8 in. hair switches for base line condition by using the Instron machine to determine a baseline force. The operator then applies a measured amount of shampoo and/or conditioner to a hair switch, distributes the product evenly through the switch. For conditioner testing, it is preferred to prewash the hair switch with a shampoo, rinse and then apply the conditioner. The wet forces are then measured as the product is rinsed using the Instron machine. Each test product is applied to a total of 4 switches. The data is then analyzed using standard statistical methods.

### H. Dry Conditioning Tests

This inter-fiber friction test determines the amount of friction on the hair provided by shampoo as measured by the force required to move hair up and down pass each other. This method emulates the motion of rubbing hair between the thumb and index finger in an up and down direction the treated hair switch. The operator ranks and balances the 4 g, 8 in. hair switches for base line condition by using an Instron machine. The operator then applies a measured amount of hair care composition to a hair switch, distributes the product evenly through the switch and rinses as per the protocol. For conditioner testing, it is preferred to prewash the hair switch with a shampoo, rinse and then apply the conditioner. Wet switches are then allowed to dry overnight and evaluated the next day for friction force using the Instron machine. Each test product is applied to a total of 4 switches. The data is then analyzed using standard statistical methods.

### EXAMPLES

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

Non-limiting examples of product formulations disclosed in the present specification are summarized below.

### Synthetic Example 1: Synthesis of Metathesized Canola Oil

Prior to the metathesis reaction, the RBD (refined, bleached, and deodorized) canola oil is pre-treated by mixing the oil with 2% (by weight) bleaching clay (Filtrol F-160, BASF, Florham Park, N.J.) and heating to 120° C. with a nitrogen sweep for 1.5 hours. The oil is cooled to room temperature, filtered through a bed of Celite® 545 diatomaceous earth (EMD, Billerica, Mass.), and stored under inert gas until ready to use.

To a round-bottomed flask, the oil is added and sub-surface sparged with inert gas while mixing and heating to 55° C. The catalyst is dissolved in 1,2-dichloroethane ([107-06-2], EMD, Billerica, Mass.) that is stored over 4 Å molecular sieves and sub-surface sparged with inert gas prior to use. After catalyst addition to the reaction flask, a vacuum is applied to remove volatile olefins that are generated. After the defined reaction time, the vacuum is broken and the metathesized unsaturated polyol ester is cooled to room temperature.

The metathesized canola oil is diluted in hexanes ([110-54-3], EMD, Billerica, Mass.). To the diluted material, 2% bleaching clay (Filtrol F-160, BASF, Florham Park, N.J.) is added and mixed for ˜6 hours. The oil is filtered through a bed of Celite® 545 diatomaceous earth. The oil is treated a second time with 2% bleaching clay (Filtrol F-160, BASF, Florham Park, N.J.) for ˜6 hours. The oil is filtered through a bed of Celite® 545 diatomaceous earth and then rotary evaporated to concentrate.

The metathesized canola oil is then passed through a wipe film evaporator at 180° C. and <0.5 Torr vacuum to remove olefins up to and including C-18 chain lengths. Representative examples are summarized in Table 4 below.

TABLE 4

| | Pretreated | | Cata- | Max | Max | Reaction |
|---|---|---|---|---|---|---|
| Exam- | Canola Oil | Cata- | lyst | Temperature | Vacuum | Time |
| ple | (g)^{a} | lyst | (g) | (° C.) | (Torr) | (min) |
| 1A | 500 | 1^{b} | 0.25 | 61 | 7.9 | 87 |
| 1B | 500 | 2^{c} | 0.25 | 62 | 0.6 | 45 |
| 1C | 500 | 2^{c} | 0.025 | 90 | 0.1 | 60 |
| 1D | 13,000 | 2^{c} | 0.65 | 80 | >1 | 120 |

^{a}Canola oil from J. Edwards, Braintree, MA.
^{b}Catalyst 1 is Tricyclohexylphosphine [4,5-dimethyl-1,3-bis(2,4,6-trimethylphenyl)imidazol-2-ylidene] [2-thienylmethylene]ruthenium (II) dichloride [1190427-50-9] available as CatMETium RF-3 from Evonik Corporation, Parsippany, NJ.
^{c}Catalyst 2 is Tricyclohexylphosphine[1,3-bis(2,4,6-trimethylphenyl)imidazol-2-ylidene][2-thienylmethylene] ruthenium(II) dichloride [1190427-49-6] available as CatMETium RF-2 from Evonik Corporation, Parsippany, NJ.

Synthetic Examples 1A, 1B, 1C and 1D are analyzed for weight average molecular weight (Mw), and free hydrocarbon content, and samples 1A and 1B are analyzed for iodine value and oligomer index, using methods described previously, and are found to approximately have the following values:

| | | | Free | |
|---|---|---|---|---|
| Synthetic | Mw | Iodine Value | Hydrocarbon | Oligomer |
| Example | (g/mol) | (cg/g) | content (wt %) | Index |
| 1A | 5,400 | 85 | 0.5 | 0.05 |
| 1B | 3,900 | 85 | 0.5 | 0.04 |
| 1C | 21,000 | Not measured | 0.5 | Not measured |
| 1D | 10,000 | Not measured | 0.2 | Not measured |

### Synthetic Example 2: Remetathesis of Metathesized Unsaturated Polyol Ester

Metathesized canola oil, sufficiently stripped of residual olefins (176.28 g from Example 1A) is blended with pretreated canola oil (350.96 g, pretreated as described in Example 1) in a round-bottomed flask. The blend is sub-surface sparged with inert gas while mixing and heating to 55° C. The catalyst is dissolved in 1,2-dichloroethane ([107-06-2], EMD, Billerica, Mass.) that is stored over 4 Å molecular sieves and sub-surface sparged with inert gas prior to use. After catalyst addition to the reaction flask, a vacuum is applied to remove volatile olefins that are generated. After ˜100 minutes of reaction time, the vacuum is broken and the metathesized unsaturated polyol ester is cooled to room temperature.

The metathesized canola oil is diluted in hexanes ([110-54-3], EMD, Billerica, Mass.). To the diluted material, 2% bleaching clay (Filtrol F-160, BASF, Florham Park, N.J.) is added and mixed for ˜6 hours. The oil is filtered through a bed of Celite® 545 diatomaceous earth. The oil is treated a second time with 2% bleaching clay (Filtrol F-160, BASF, Florham Park, N.J.) for ˜6 hours. The oil is filtered through a bed of Celite® 545 diatomaceous earth and then rotary evaporated to concentrate.

The remetathesized canola oil is then passed through a wipe film evaporator at 180° C. and <0.5 Torr vacuum to remove olefins up to and including C-18 chain lengths. A representative example is summarized in Table 5 below.

TABLE 5

| | | | Max | Max |
|---|---|---|---|---|
| Synthetic | Oil Blend | Catalyst^{a} | Temperature | Vacuum |
| Example | (g) | (g) | (° C.) | (Torr) |
| 2 | 500 | 0.27 | 65 | 0.2 |

^{a}Tricyclohexylphosphine [4,5-dimethyl-1,3-bis(2,4,6-trimethylphenyl)imidazol-2-ylidene] [2-thienylmethylene]ruthenium (II) dichloride [1190427-50-9] available as CatMETium RF-3 from Evonik Corporation, Parsippany, NJ.

The sample 2 is analyzed for weight average molecular weight, iodine value, free hydrocarbon content and oligomer index, using methods described previously, and is found to approximately have the following values:

| | | | Free | |
|---|---|---|---|---|
| Synthetic | | Iodine Value | Hydrocarbon | Oligomer |
| Example | Mw (g/mol) | (cg/g) | content (wt %) | Index |
| 2 | 13,000 | 80 | 0.5 | 0.07 |

### Synthetic Example 3: Synthesis of Metathesized Unsaturated Polyol Esters

Prior to the metathesis reaction, the RBD (refined, bleached, and deodorized) oil is pre-treated by mixing the oil with 2% (by weight) bleaching clay (Filtrol F-160, BASF, Florham Park, N.J.) and heating to 120° C. with a nitrogen sweep for 1.5 hours. The oil is cooled to room temperature, filtered through a bed of Celite® 545 diatomaceous earth (EMD, Billerica, Mass.), and stored under inert gas until ready to use.

To a round-bottomed flask, the oil is added and sub-surface sparged with inert gas while mixing and heating to 55° C. The catalyst is dissolved in 1,2-dichloroethane ([107-06-2], EMD, Billerica, Mass.) that is stored over 4 Å molecular sieves and sub-surface sparged with inert gas prior to use. After catalyst addition to the reaction flask, a vacuum is applied to remove volatile olefins that are generated. After ˜4 hours reaction time, the vacuum is broken and the metathesized unsaturated polyol ester is cooled to room temperature.

The metathesized oil is diluted in hexanes ([110-54-3], EMD, Billerica, Mass.). To the diluted material, 2% bleaching clay (Filtrol F-160, BASF, Florham Park, N.J.) is added and mixed for ˜6 hours. The metathesized oil is filtered through a bed of Celite® 545 diatomaceous earth. The metathesized oil is treated a second time with 2% bleaching clay (Filtrol F-160, BASF, Florham Park, N.J.) for ˜6 hours. The metathesized oil is filtered through a bed of Celite® 545 diatomaceous earth and then rotary evaporated to concentrate.

The metathesized unsaturated polyol ester is then passed through a wipe film evaporator at 180° C. and <0.5 Torr vacuum to remove olefins up to and including C-18 chain lengths. Representative examples are summarized in Table 6 below.

TABLE 6

| | Starting | | | Max | Max |
|---|---|---|---|---|---|
| Synthetic | unsaturated | Pretreated Oil | | Temperature | Vacuum |
| Example | polyol ester | (g) | Catalyst^{a} (g) | (° C.) | (Torr) |
| 3A | High erucic | 500 | 0.25 | 61 | 7.9 |
| | acid | | | | |
| | rapeseed oil | | | | |
| 3B | Blend of | 500 (250 g | 0.25 | 61 | 7.9 |
| | High erucic | HEAR oil and | | | |
| | acid | 250 g canola oil) | | | |
| | rapeseed oil | | | | |
| | and canola | | | | |
| | oil, 50/50 by | | | | |
| | weight | | | | |
| 3C | High oleic | 500 | 0.25 | 61 | 7.9 |
| | soybean oil | | | | |

^{a}Tricyclohexylphosphine [4,5-dimethyl-1,3-bis(2,4,6-trimethylphenyl)imidazol-2-ylidene] [2-thienylmethylene]ruthenium (II) dichloride [1190427-50-9] available as CatMETium RF-3 from Evonik Corporation, Parsippany, NJ.

### Synthetic Example 4

Hydrogenations are performed in a T316 stainless steel, 600 ml Parr reactor (Model Number 4563) containing internal cooling coils and a stir shaft with 2 impellers comprised of 4 blades each.

The metathesized unsaturated polyol ester (approximately 200 g) is dissolved in hexanes (120 ml, [110-54-3], EMD, Billerica Ma). To this solution is added a slurry of Nickel on Silica (20 g, [7440-02-0], Catalog #28-1900, Strem Chemicals, Inc., Newburyport, Mass.). The slurried mixture is transferred via vacuum to the Parr reactor. The mixture is degassed with several vacuum/nitrogen fill cycles. Then with stirring (800-900 rpm), hydrogen gas (550-650 psig, [1333-74-0], UHP grade, Wright Brothers, Inc., Montgomery, Ohio) is charged to the reactor. The reaction is heated at 150° C. and hydrogen gas pressure reduction monitored until constant (˜12 hours).

The reaction is cooled to 60° C. and drained from the reactor. The reactor is rinsed with methyl tert-butyl ether ([1634-04-4], EMD, Billerica, Mass.) and combined with the solid hydrogenated metathesized polyol ester. A hot filtration is then performed to remove the catalyst, followed by vacuum to remove all residual solvent. Fully hydrogenated materials are obtained using the method above. Lower hydrogenation levels are obtained by decreasing the reaction temperature to 125° C. using 5 grams of catalyst and reducing the reaction time and hydrogen consumed. Iodine Value (IV) is measured, as described elsewhere.

### Synthetic Example 5

A round bottom flask is charged with palm oil (approximately 500 g), heated to 60° C. to melt the oil and sparged with nitrogen for one hour using a gas dispersion tube. The nitrogen sparge tube is lifted above the surface of the liquid to blanket the oil and Filtrol F-160 (2%) is charged to the flask under rapid agitation and the reactor is heated to 120° C. for one hour. The flask is cooled to 90° C. and toluene is added to reduce the nonvolatile content to 70%. The solution is filtered with a Buchner funnel containing a pile of Whatman Grade 1 filter paper, glass microfiber pad, filter paper and Celite 454.

Treated palm oil solution is transferred to a 4 neck round bottom flask equipped with a central mechanical agitator, thermometer, glass stopper and a connecting tube with a vacuum take-off and a chilled receiver for metathesis reaction. The flask is sparged with dry nitrogen for 1 hour and the flask is heated to 90° C. A separate oven-dried flask is charged with CatMETium RF2 catalyst (50 ppm) and 1,2-Dichloroethane (kept over sieves, sparged for 45 min with nitrogen). The nitrogen sparge tube is raised to blanket the oil and the catalyst solution is added to the 90° C. palm oil using a cannula. Vacuum is immediately applied through a chilled 2 L trap and reaches 2 mm within a few minutes ultimately reaching 0.11 mm as distillate removal slows. Vacuum and temperature are held for 4 hours. The flask is cooled to room temperature.

The catalyst is removed by stirring the palm polyoil solution with Filtrol F-160 (2%) at 50° C. overnight followed by filtration using a Buchner funnel containing a pile of filter paper, a piece of glass wool pad, a piece of filter paper and Celite 454. This treatment is performed twice. High boiling olefin such as 9-octadecene and residual toluene are removed by a vacuum stripping procedure using a 3 neck flask containing a thermometer, mechanical agitation and a connecting tube with a vacuum takeoff and chilled receiver. The temperature is set for 130° C. Toluene is removed quickly and olefin begins to be removed at about 105° C. Vacuum improves as the olefin removal slows and reaches 0.06 mm when olefin removal is very slow. The final palm polyoil is discharged at 60° C.

The palm polyoil of Synthetic Example 5 is analyzed for weight average molecular weight, iodine value, free hydrocarbon content, and oligomer index using methods described previously, and is found to approximately have the following values:

| | | | Free | |
|---|---|---|---|---|
| Synthetic | | Iodine Value | Hydrocarbon | Oligomer |
| Example | Mw (g/mol) | (cg/g) | content (wt %) | Index |
| 5 | 4000 | 43 | 1.6 | 0.13 |

### Synthetic Example 6

The metathesis monomers, dimers, trimers, tetramers, pentamers, and higher order oligomers from the product in Synthetic Example 2 are fully separated by SFC using the method described above. The individual SFC fractions are collected and trimers, tetramers, and higher order oligomers are combined. The oligomer index of this sample is about 1.

Composition Examples 1 through 32 below are representative of hair care compositions of the present invention. The exemplified compositions can be prepared by conventional formulation and mixing techniques. Comparative Examples A through G below are not representative of hair care compositions of the present invention. The list of footnoted ingredients for Examples 1 through 32 and Comparative Examples A through G is after the table summarizing Comparative Examples A through G. A key difference between the inventive examples and corresponding comparative examples is the properties of metathesized oils, as represented in Table 7 below for those specific materials. The comparative material has a weight average molecular weight of less than 5,000 Daltons, an Iodine value of less than 8 and a free hydrocarbon of 6% or more. In contrast, all inventive materials have one or more of i) a free hydrocarbon content of from 0-5%, ii) a weight average molecular weight of from 5,000-50,000 Daltons; iii) an iodine value of from 8-200.

It will be appreciated that other modifications of the hair care composition within the skill of those in the hair care formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

TABLE 7

| | Metathesized oils | Mw | IV | Free hydrocarbons, % |
|---|---|---|---|---|
| Comparative | Hydrogenated soy | 3,900 | 4.4 | 6-11 |
| | polyglycerides (and) C₁₅₋₂₃ | | | |
| | alkane¹ | | | |
| Inventive | Metathesized canola oil² | 3,900 | 85 | 0.5 |
| | Metathesized canola oil³ | 21,000 | Not | 0.5 |
| | | | measured | |
| | Metathesized canola oil⁴ | 10,000 | Not | 0.2 |
| | | | measured | |
| | Metathesized Palm oil⁵ | 4,000 | 43 | 1.6 |

¹Elevance Smooth CS-110, available from Elevance Renewable Sciences, Woodridge, IL.
²Synthetic Example 1B in Table 4.
³Synthetic Example 1C in Table 4.
⁴Synthetic Example 1D in Table 4.
⁵Synthetic Example 5 above.

### Rinse-Off Conditioner Composition Examples 1-32

| Components | Ex. 1 | Ex. 2 | Ex. 3 | Ex. 4 | Ex. 5 | Ex. 6 |
|---|---|---|---|---|---|---|
| Water | q.s. | q.s. | q.s. | q.s. | q.s. | q.s. |
| BTMS¹ | 2.3 | 2.3 | 2.3 | 2.3 | 2.3 | 2.3 |
| Cetyl alcohol³ | 1.1 | 1.1 | 1.1 | 1.1 | 1.1 | 1.1 |
| Stearyl alcohol⁴ | 2.8 | 2.8 | 2.8 | 2.8 | 2.8 | 2.8 |
| Aminosilicone⁵ | — | — | 0.5 | 0.5 | — | — |
| Metathesized canola oil ⁶ | — | 1.0 | — | — | 2.0 | — |
| Metathesized canola oil ⁷ | — | — | — | 1.0 | — | — |
| Metathesized canola oil ⁸ | — | — | — | — | — | — |
| Metathesized Palm oil⁹ | 1.0 | — | 0.5 | — | — | 2.0 |
| Perfume | 0.5 | 0.5 | 0.5 | 0.5 | 0.5 | 0.5 |
| Preservatives, pH, | Up to 5% | Up to 5% | Up to 5% | Up to 5% | Up to 5% | Up to 5% |
| viscosity adjustment | | | | | | |
| Components | Ex. 7 | Ex. 8 | Ex. 9 | Ex. 10 | Ex. 11 | Ex. 12 |
| Water | q.s. | q.s. | q.s. | q.s. | q.s. | q.s. |
| BTMAC² | 2.8 | 2.8 | 2.8 | 2.8 | 2.8 | 2.8 |
| Cetyl alcohol³ | 1.8 | 1.8 | 1.8 | 1.8 | 1.8 | 1.8 |
| Stearyl alcohol⁴ | 4.6 | 4.6 | 4.6 | 4.6 | 4.6 | 4.6 |
| Metathesized canola oil ⁶ | 1.0 | — | — | — | — | — |
| Metathesized canola oil ⁷ | — | 1.0 | — | — | — | — |
| Metathesized canola oil ⁸ | — | — | 1.0 | — | — | 2.0 |
| Metathesized Palm oil⁹ | — | — | — | 1.0 | 2.0 | — |
| Aminosilicone⁵ | — | — | 0.75 | 0.75 | — | — |
| Perfume | 0.5 | 0.5 | 0.5 | 0.5 | 0.5 | 0.5 |
| Preservatives, pH, | Up to 5% | Up to 5% | Up to 5% | Up to 5% | Up to 5% | Up to 5% |
| viscosity adjustment | | | | | | |

| Components | Ex. 13 | Ex. 14 | Ex. 15 | Ex. 16 |
|---|---|---|---|---|
| Water | q.s. | q.s. | q.s. | q.s. |
| BTMS¹ | 3.76 | 3.76 | 3.76 | 3.76 |
| Cetyl alcohol³ | 1.3 | 1.3 | 1.3 | 1.3 |
| Stearyl alcohol⁴ | 3.2 | 3.2 | 3.2 | 3.2 |
| Metathesized canola oil⁸ | — | — | 1.0 | 1.0 |
| Metathesized Palm oil⁹ | 1.0 | 1.0- | — | — |
| Perfume | 0.5 | 0.5 | 0.5 | 0.5 |
| Preservatives, pH, viscosity | Up to 5% | Up to 5% | Up to 5% | Up to 5% |
| adjustment | | | | |
| Deposition Aid polymer¹⁰ | 0.5 | — | 0.5 | — |

| Components | Ex. 17 | Ex. 18 | Ex. 19 | Ex. 20 | Ex. 21 | Ex. 22 | Ex. 23 | Ex. 24 |
|---|---|---|---|---|---|---|---|---|
| Water | q.s. | q.s. | q.s. | q.s. | q.s. | q.s. | q.s. | q.s. |
| SAPDMA¹⁰ | 1.95 | 1.95 | 1.95 | 1.95 | 3.24 | 3.24 | 3.24 | 3.24 |
| Cetyl alcohol³ | 1.68 | 1.68 | 1.68 | 1.68 | 4.25 | 4.25 | 4.25 | 4.25 |
| Stearyl alcohol⁴ | 2.90 | 2.90 | 2.90 | 2.90 | 2.93 | 2.93 | 2.93 | 2.93 |
| Metathesized canola oil⁸ | 0.5 | — | 1.0 | — | 1.0 | — | 2.0 | — |
| Metathesized Palm oil⁹ | — | 0.5 | — | 1.0 | — | 1.0 | — | 2.0 |
| Perfume | 0.5 | 0.5 | 0.5 | 0.5 | 0.5 | 0.5 | 0.5 | 0.5 |
| Glyceryl Monooleate¹¹ | 0.0085 | 0.0085 | 0.017 | 0.017 | 0.017 | 0.017 | 0.034 | 0.034 |
| Polysorbate 20¹² | 0.0165 | 0.0165 | 0.033 | 0.033 | 0.033 | 0.033 | 0.066 | 0.066 |
| Preservatives, pH, viscosity | Up to 5% | Up to 5% | Up to 5% | Up to 5% | Up to 5% | Up to 5% | Up to 5% | Up to 5% |
| adjustment | | | | | | | | |

In Composition Examples 17-24, the metathesized oils are emulsified with Glyceryl monooleate and Polysorbate 20 to a median particle size of about 1.2 microns prior to incorporation to the conditioner.

| Components | Ex. 25 | Ex. 26 | Ex. 27 | Ex. 28 | Ex. 29 | Ex. 30 | Ex. 31 | Ex. 32 |
|---|---|---|---|---|---|---|---|---|
| Water | q.s. | q.s. | q.s. | q.s. | q.s. | q.s. | q.s. | q.s. |
| SAPDMA¹⁰ | 1.95 | 1.95 | 1.95 | 1.95 | 3.24 | 3.24 | 3.24 | 3.24 |
| Cetyl alcohol³ | 1.68 | 1.68 | 1.68 | 1.68 | 4.25 | 4.25 | 4.25 | 4.25 |
| Stearyl alcohol⁴ | 2.90 | 2.90 | 2.90 | 2.90 | 2.93 | 2.93 | 2.93 | 2.93 |
| Metathesized canola oil⁸ | 0.5 | — | 1.0 | — | 1.0 | — | 2.0 | — |
| Metathesized Palm oil⁹ | — | 0.5 | — | 1.0 | — | 1.0 | — | 2.0 |
| Perfume | 0.5 | 0.5 | 0.5 | 0.5 | 0.5 | 0.5 | 0.5 | 0.5 |
| Preservatives, pH, viscosity | Up to 5% | Up to 5% | Up to 5% | Up to 5% | Up to 5% | Up to 5% | Up to 5% | Up to 5% |
| adjustment | | | | | | | | |

### Comparative Examples

| | Comp | Comp | Comp | Comp | Comp | Comp | Comp |
|---|---|---|---|---|---|---|---|
| Components | Ex. A | Ex. B | Ex. C | Ex. D | Ex. E | Ex. F | Ex. G |
| Water | q.s. | q.s. | q.s. | q.s. | q.s. | q.s. | q.s. |
| SAPDMA¹⁰ | 1.95 | 1.95 | 1.95 | 1.95 | 1.95 | 3.24 | 3.24 |
| Cetyl alcohol³ | 1.68 | 1.68 | 1.68 | 1.68 | 1.68 | 4.25 | 4.25 |
| Stearyl alcohol⁴ | 2.90 | 2.90 | 2.90 | 2.90 | 2.90 | 2.93 | 2.93 |
| Hydrogenated soy | 0.5 | — | — | — | — | — | — |
| polyglycerides (and) | | | | | | | |
| C₁₅₋₂₃ alkane¹³ | | | | | | | |
| Hydrogenated soybean | — | 0.5 | 0.5 | 1.0 | 1.0 | 2.0 | 2.0 |
| oil (and) Hydrogenated | | | | | | | |
| soy polyglycerides (and) | | | | | | | |
| C₁₅₋₂₃ alkane¹⁴ | | | | | | | |
| Perfume | 0.5 | 0.5 | 0.5 | 0.5 | — | — | — |
| Glyceryl Monooleate¹¹ | — | — | 0.0085 | — | 0.017 | — | 0.034 |
| Polysorbate 20¹² | — | — | 0.0165 | — | 0.033 | — | 0.066 |
| Preservatives, pH, viscosity | Up to 5% | Up to 5% | Up to 5% | Up to 5% | Up to 5% | Up to 5% | Up to 5% |
| adjustment | | | | | | | |

In Comparative Examples C, E and G, the metathesized oil is emulsified with Glyceryl monooleate and Polysorbate 20 to a median particle size of about 1.2 microns prior to incorporation to the conditioner.

### Footnotes for Composition Examples 1-32 and Comparative Examples A-G:

¹ Behenyltrimethylammonium methylsulfate, from Feixiang
² Behenyltrimethylammonium chloride, Genamin KDMP, from Clariant
³ Cetyl alcohol, from P&G
⁴ Stearyl alcohol, from P&G
⁵ Y-14945; 10,000 cps aminodimethicone, from Momentive
⁶ Synthetic Example 1B in Table 4
⁷ Synthetic Example 1C in Table 4
⁸ Synthetic Example 1D in Table 4
⁹ Metathesized palm polyol ester of Synthetic Example 5
¹⁰ Stearamidopropyldimethylamine (LEXAMINE S-13), from BASF
¹¹ MONOMULS 90-O 18, from BASF
¹² Polysorbate 20, from Croda
¹³ Elevance Smooth CS-110, from Elevance Renewable Sciences
¹⁴ Elevance Soft CG-100, from Elevance Renewable Sciences

The hair care composition may be presented in typical hair care formulations. They may be in the form of solutions, dispersion, emulsions, powders, talcs, encapsulated spheres, spongers, solid dosage forms, foams, and other delivery mechanisms. The compositions of the embodiments of the present invention may be hair tonics, leave-on hair products such as treatment and styling products, rinse-off hair products such as shampoos, and any other form that may be applied to hair.

In one embodiment, the hair care compositions may be provided in the form of a porous, dissolvable solid structure, such as those disclosed in U.S. Patent Application Publication Nos. 2009/0232873; and 2010/0179083, which are incorporated herein by reference in their entirety. As described in these references, such dissolvable solid structure embodiments will typically have a water content well below the at least about 20% aqueous carrier element of certain embodiments described above.

The hair care compositions are generally prepared by conventional methods such as those known in the art of making the compositions. Such methods typically involve mixing of the ingredients in one or more steps to a relatively uniform state, with or without heating, cooling, application of vacuum, and the like. The compositions are prepared such as to optimize stability (physical stability, chemical stability, photostability) and/or delivery of the active materials. The hair care composition may be in a single phase or a single product, or the hair care composition may be in a separate phases or separate products. If two products are used, the products may be used together, at the same time or sequentially. Sequential use may occur in a short period of time, such as immediately after the use of one product, or it may occur over a period of hours or days.

The composition provided by the formula above is made by combining such ingredients in accordance with the method of making provided in this specification.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

### Examples/Combinations

A. A hair care composition comprising: (a) from about 0.05% to about 15%, by weight of said hair care composition, of one or more metathesized unsaturated polyol esters, said metathesized unsaturated polyol ester having one or more of the following properties: (i) a free hydrocarbon content, based on total weight of metathesized unsaturated polyol ester, of from about 0% to about 5%; (ii) a weight average molecular weight of from about 5,000 Daltons to about 50,000 Daltons; (iii) an iodine value of from about 30 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.
B. The hair care composition of paragraph A, wherein the metathesized unsaturated polyol ester has a free hydrocarbon content, based on total weight of metathesized unsaturated polyol ester, of from about 0% to about 5%.
C. The hair care composition of paragraph A or B, wherein the metathesized unsaturated polyol ester has a free hydrocarbon content, based on total weight of metathesized unsaturated polyol ester, of from about 0.1% to about 4%.
D. The hair care composition of any one of paragraphs A-C, wherein the metathesized unsaturated polyol ester has a weight average molecular weight of from about 5,000 Daltons to about 50,000 Daltons.
E. The hair care composition of any one of paragraphs A-D, wherein the metathesized unsaturated polyol ester has a weight average molecular weight of from about 6,000 Daltons to about 30,000 Daltons.
F. The hair care composition of any one of paragraphs A-E, wherein the metathesized unsaturated polyol ester has a weight average molecular weight of from about 5,000 Daltons to about 50,000 Daltons.
G. The hair care composition of any one of paragraphs A-F, wherein the metathesized unsaturated polyol ester has an iodine value of from about 30 to about 200.
H. The hair care composition of any one of paragraphs A-G, wherein the metathesized unsaturated polyol ester has an iodine value of from about 30 to about 120.
I. The hair care composition of any one of paragraphs A-H, wherein the metathesized unsaturated polyol ester has an iodine value of from about 30 to about 200.
J. The hair care composition of any one of paragraphs A-I, wherein the metathesized unsaturated polyol ester has a free hydrocarbon content, based on total weight of metathesized unsaturated polyol ester, of from about 0% to about 5%.
K. The hair care composition of any one of paragraphs A-J, wherein said metathesized unsaturated polyol ester is selected from the group consisting of metathesized abyssinian oil, metathesized almond oil, metathesized apricot oil, metathesized apricot kernel oil, metathesized argan oil, metathesized avocado oil, metathesized babassu oil, metathesized baobab oil, metathesized black cumin oil, metathesized black currant oil, metathesized borage oil, metathesized camelina oil, metathesized carinata oil, metathesized canola oil, metathesized castor oil, metathesized cherry kernel oil, metathesized coconut oil, metathesized corn oil, metathesized cottonseed oil, metathesized echium oil, metathesized evening primrose oil, metathesized flax seed oil, metathesized grape seed oil, metathesized grapefruit seed oil, metathesized hazelnut oil, metathesized hemp seed oil, metathesized jatropha oil, metathesized jojoba oil, metathesized kukui nut oil, metathesized linseed oil, metathesized macadamia nut oil, metathesized meadowfoam seed oil, metathesized moringa oil, metathesized neem oil, metathesized olive oil, metathesized palm oil, metathesized palm kernel oil, metathesized peach kernel oil, metathesized peanut oil, metathesized pecan oil, metathesized pennycress oil, metathesized perilla seed oil, metathesized pistachio oil, metathesized pomegranate seed oil, metathesized pongamia oil, metathesized pumpkin seed oil, metathesized raspberry oil, metathesized red palm olein, metathesized rice bran oil, metathesized rosehip oil, metathesized safflower oil, metathesized seabuckthorn fruit oil, metathesized sesame seed oil, metathesized shea glein, metathesized sunflower oil, metathesized soybean oil, metathesized tonka bean oil, metathesized tung oil, metathesized walnut oil, metathesized wheat germ oil, metathesized high oleoyl soybean oil, metathesized high oleoyl sunflower oil, metathesized high oleoyl safflower oil, metathesized high erucic acid rapeseed oil, metathesized lard, metathesized tallow, metathesized poultry fat, metathesized yellow grease, metathesized fish oil, and mixtures thereof.
L. A hair care composition comprising: a) a metathesized unsaturated polyol ester, said metathesized unsaturated polyol ester having a weight average molecular weight of from about 2,000 Daltons to about 50,000 Daltons; and one or more of the following properties: (i) a free hydrocarbon content, based on total weight of metathesized unsaturated polyol ester, of from about 0% to about 5%; or (ii) an iodine value of from about 8 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.
M. The hair care composition of paragraph L, wherein said metathesized unsaturated polyol ester has a free hydrocarbon content, based on total weight of metathesized unsaturated polyol ester, of from about 0% to about 5%.
N. The hair care composition of paragraph L or M, wherein said metathesized unsaturated polyol ester has a free hydrocarbon content, based on total weight of metathesized unsaturated polyol ester, of from about 0.1% to about 4%.
O. The hair care composition of any one of paragraphs L-N, wherein said metathesized unsaturated polyol ester has an iodine value of from about 8 to about 200.
P. The hair care composition of any one of paragraphs L-O, wherein said metathesized unsaturated polyol ester has an iodine value of from about 30 to about 120.
Q. The hair care composition of any one of paragraphs L-P, wherein said metathesized unsaturated polyol ester has a weight average molecular weight of from about 4,000 Daltons to about 30,000 Daltons.
R. The hair care composition of any one of paragraphs L-Q, comprising a metathesized unsaturated polyol ester, said metathesized unsaturated polyol ester having i) a weight average molecular weight of from about 2,000 Daltons to about 30,000 Daltons; ii) a free hydrocarbon content, based on total weight of metathesized unsaturated polyol ester, of from about 0.1 to about 3%; and (iii) an iodine value of from about 30 to about 120.
S. The hair care composition of any one of paragraphs L-R, wherein said metathesized unsaturated polyol ester is selected from the group consisting of metathesized abyssinian oil, metathesized almond oil, metathesized apricot oil, metathesized apricot kernel oil, metathesized argan oil, metathesized avocado oil, metathesized babassu oil, metathesized baobab oil, metathesized black cumin oil, metathesized black currant oil, metathesized borage oil, metathesized camelina oil, metathesized carinata oil, metathesized canola oil, metathesized castor oil, metathesized cherry kernel oil, metathesized coconut oil, metathesized corn oil, metathesized cottonseed oil, metathesized echium oil, metathesized evening primrose oil, metathesized flax seed oil, metathesized grape seed oil, metathesized grapefruit seed oil, metathesized hazelnut oil, metathesized hemp seed oil, metathesized jatropha oil, metathesized jojoba oil, metathesized kukui nut oil, metathesized linseed oil, metathesized macadamia nut oil, metathesized meadowfoam seed oil, metathesized moringa oil, metathesized neem oil, metathesized olive oil, metathesized palm oil, metathesized palm kernel oil, metathesized peach kernel oil, metathesized peanut oil, metathesized pecan oil, metathesized pennycress oil, metathesized perilla seed oil, metathesized pistachio oil, metathesized pomegranate seed oil, metathesized pongamia oil, metathesized pumpkin seed oil, metathesized raspberry oil, metathesized red palm olein, metathesized rice bran oil, metathesized rosehip oil, metathesized safflower oil, metathesized seabuckthorn fruit oil, metathesized sesame seed oil, metathesized shea glein, metathesized sunflower oil, metathesized soybean oil, metathesized tonka bean oil, metathesized tung oil, metathesized walnut oil, metathesized wheat germ oil, metathesized high oleoyl soybean oil, metathesized high oleoyl sunflower oil, metathesized high oleoyl safflower oil, metathesized high erucic acid rapeseed oil, metathesized lard, metathesized tallow, metathesized poultry fat, metathesized yellow grease, metathesized fish oil, and mixtures thereof.
T. The hair care composition according to any one of paragraphs A-S, wherein said hair care composition further comprises from about 0.03% to about 8% of a deposition polymer which is a copolymer comprising a vinyl monomer (A) with a carboxyl group in the structure; and a vinyl monomer (B) expressed by the following formula (1):

CH₂═C(R¹)—CO—X-(Q-O)ᵣ—R² (1)
wherein R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O)ᵣ—R², the number of atoms bonded in a straight chain is 70 or less; and wherein the vinyl monomer (A) is contained at a level of from about 10 mass % to about 50 mass %, and the vinyl monomer (B) is contained at level of from about 50 mass % to about 90 mass %.

What is claimed is:

1. A hair care composition comprising: (a) from about 0.05% to about 15%, by weight of said hair care composition, of one or more metathesized unsaturated polyol esters, said metathesized unsaturated polyol ester having one or more of the following properties: (i) a free hydrocarbon content, based on total weight of metathesized unsaturated polyol ester, of from about 0% to about 5%; (ii) a weight average molecular weight of from about 5,000 Daltons to about 50,000 Daltons; (iii) an iodine value of from about 30 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

2. The hair care composition of claim 1 wherein the metathesized unsaturated polyol ester has a free hydrocarbon content, based on total weight of metathesized unsaturated polyol ester, of from about 0% to about 5%.

3. The hair care composition of claim 2 wherein the metathesized unsaturated polyol ester has a free hydrocarbon content, based on total weight of metathesized unsaturated polyol ester, of from about 0.1% to about 4%.

4. The hair care composition of claim 3 wherein the metathesized unsaturated polyol ester has a weight average molecular weight of from about 5,000 Daltons to about 50,000 Daltons.

5. The hair care composition of claim 4 wherein the metathesized unsaturated polyol ester has a weight average molecular weight of from about 6,000 Daltons to about 30,000 Daltons.

6. The hair care composition of claim 1 wherein the metathesized unsaturated polyol ester has a weight average molecular weight of from about 5,000 Daltons to about 50,000 Daltons.

7. The hair care composition of claim 6 wherein the metathesized unsaturated polyol ester has an iodine value of from about 30 to about 200.

8. The hair care composition of claim 7 wherein the metathesized unsaturated polyol ester has an iodine value of from about 30 to about 120.

9. The hair care composition of claim 1 wherein the metathesized unsaturated polyol ester has an iodine value of from about 30 to about 200.

10. The hair care composition of claim 9 wherein the metathesized unsaturated polyol ester has a free hydrocarbon content, based on total weight of metathesized unsaturated polyol ester, of from about 0% to about 5%.

11. The hair care composition according to claim 1 wherein said metathesized unsaturated polyol ester is selected from the group consisting of metathesized abyssinian oil, metathesized almond oil, metathesized apricot oil, metathesized apricot kernel oil, metathesized argan oil, metathesized avocado oil, metathesized babassu oil, metathesized baobab oil, metathesized black cumin oil, metathesized black currant oil, metathesized borage oil, metathesized camelina oil, metathesized carinata oil, metathesized canola oil, metathesized castor oil, metathesized cherry kernel oil, metathesized coconut oil, metathesized corn oil, metathesized cottonseed oil, metathesized echium oil, metathesized evening primrose oil, metathesized flax seed oil, metathesized grape seed oil, metathesized grapefruit seed oil, metathesized hazelnut oil, metathesized hemp seed oil, metathesized jatropha oil, metathesized jojoba oil, metathesized kukui nut oil, metathesized linseed oil, metathesized macadamia nut oil, metathesized meadowfoam seed oil, metathesized moringa oil, metathesized neem oil, metathesized olive oil, metathesized palm oil, metathesized palm kernel oil, metathesized peach kernel oil, metathesized peanut oil, metathesized pecan oil, metathesized pennycress oil, metathesized perilla seed oil, metathesized pistachio oil, metathesized pomegranate seed oil, metathesized pongamia oil, metathesized pumpkin seed oil, metathesized raspberry oil, metathesized red palm olein, metathesized rice bran oil, metathesized rosehip oil, metathesized safflower oil, metathesized seabuckthorn fruit oil, metathesized sesame seed oil, metathesized shea glein, metathesized sunflower oil, metathesized soybean oil, metathesized tonka bean oil, metathesized tung oil, metathesized walnut oil, metathesized wheat germ oil, metathesized high oleoyl soybean oil, metathesized high oleoyl sunflower oil, metathesized high oleoyl safflower oil, metathesized high erucic acid rapeseed oil, metathesized lard, metathesized tallow, metathesized poultry fat, metathesized yellow grease, metathesized fish oil, and mixtures thereof.

12. A hair care composition comprising: a) a metathesized unsaturated polyol ester, said metathesized unsaturated polyol ester having a weight average molecular weight of from about 2,000 Daltons to about 50,000 Daltons; and one or more of the following properties: (i) a free hydrocarbon content, based on total weight of metathesized unsaturated polyol ester, of from about 0% to about 5%; or (ii) an iodine value of from about 8 to about 200; and (b) a gel matrix phase comprising: (i) from about 0.1% to about 20% of one or more high melting point fatty compounds, by weight of said hair care composition; (ii) from about 0.1% to about 10% of a cationic surfactant system, by weight of said hair care composition; and (iii) at least about 20% of an aqueous carrier, by weight of said hair care composition.

13. The hair care composition according to claim 12 wherein said metathesized unsaturated polyol ester has a free hydrocarbon content, based on total weight of metathesized unsaturated polyol ester, of from about 0% to about 5%.

14. The hair care composition according to claim 13 wherein said metathesized unsaturated polyol ester has a free hydrocarbon content, based on total weight of metathesized unsaturated polyol ester, of from about 0.1% to about 4%.

15. The hair care composition according to claim 12 wherein said metathesized unsaturated polyol ester has an iodine value of from about 8 to about 200.

16. The hair care composition according to claim 15 wherein said metathesized unsaturated polyol ester has an iodine value of from about 30 to about 120.

17. The hair care composition according to claim 12 wherein said metathesized unsaturated polyol ester has a weight average molecular weight of from about 4,000 Daltons to about 30,000 Daltons.

18. The hair care composition according to claim 12 comprising a metathesized unsaturated polyol ester, said metathesized unsaturated polyol ester having i) a weight average molecular weight of from about 2,000 Daltons to about 30,000 Daltons; ii) a free hydrocarbon content, based on total weight of metathesized unsaturated polyol ester, of from about 0.1 to about 3%; and (iii) an iodine value of from about 30 to about 120.

19. The hair care composition according to claim 12 wherein said metathesized unsaturated polyol ester is selected from the group consisting of metathesized abyssinian oil, metathesized almond oil, metathesized apricot oil, metathesized apricot kernel oil, metathesized argan oil, metathesized avocado oil, metathesized babassu oil, metathesized baobab oil, metathesized black cumin oil, metathesized black currant oil, metathesized borage oil, metathesized camelina oil, metathesized carinata oil, metathesized canola oil, metathesized castor oil, metathesized cherry kernel oil, metathesized coconut oil, metathesized corn oil, metathesized cottonseed oil, metathesized echium oil, metathesized evening primrose oil, metathesized flax seed oil, metathesized grape seed oil, metathesized grapefruit seed oil, metathesized hazelnut oil, metathesized hemp seed oil, metathesized jatropha oil, metathesized jojoba oil, metathesized kukui nut oil, metathesized linseed oil, metathesized macadamia nut oil, metathesized meadowfoam seed oil, metathesized moringa oil, metathesized neem oil, metathesized olive oil, metathesized palm oil, metathesized palm kernel oil, metathesized peach kernel oil, metathesized peanut oil, metathesized pecan oil, metathesized pennycress oil, metathesized perilla seed oil, metathesized pistachio oil, metathesized pomegranate seed oil, metathesized pongamia oil, metathesized pumpkin seed oil, metathesized raspberry oil, metathesized red palm olein, metathesized rice bran oil, metathesized rosehip oil, metathesized safflower oil, metathesized seabuckthorn fruit oil, metathesized sesame seed oil, metathesized shea glein, metathesized sunflower oil, metathesized soybean oil, metathesized tonka bean oil, metathesized tung oil, metathesized walnut oil, metathesized wheat germ oil, metathesized high oleoyl soybean oil, metathesized high oleoyl sunflower oil, metathesized high oleoyl safflower oil, metathesized high erucic acid rapeseed oil, metathesized lard, metathesized tallow, metathesized poultry fat, metathesized yellow grease, metathesized fish oil, and mixtures thereof.

20. The hair care composition according to claim 12 wherein said hair care composition further comprises from about 0.03% to about 8% of a deposition polymer which is a copolymer comprising a vinyl monomer (A) with a carboxyl group in the structure; and a vinyl monomer (B) expressed by the following formula (1):
CH₂═C(R¹)—CO—X-(Q-O)ᵣ—R² (1)
wherein R¹ represents a hydrogen atom or a methyl group; R² represents a hydrogen atom or an alkyl group with from 1 to 5 carbon atoms, which may have a substitution group; Q represents an alkylene group with from 2 to 4 carbon atoms which may also have a substitution group; r represents an integer from 2 to 15; and X represents an oxygen atom or an NH group; and, in the following structure -(Q-O)ᵣ—R², the number of atoms bonded in a straight chain is 70 or less; and wherein the vinyl monomer (A) is contained at a level of from about 10 mass % to about 50 mass %, and the vinyl monomer (B) is contained at level of from about 50 mass % to about 90 mass %.
